SEQUENCE LISTING (38 sequences)

SEQ ID NO: 1 - DNA, 18 bp, Mus musculus
  source 1..18: mol_type = genomic DNA
cagcagcagcagcagcag

SEQ ID NO: 2 - DNA, 18 bp, Mus musculus
  source 1..18: mol_type = genomic DNA
ctgctgctgctgctgctg

SEQ ID NO: 3 - RNA, 18 nt, Mus musculus
  source 1..18: mol_type = genomic RNA
ctgctgctgctgctgctg

SEQ ID NO: 4 - DNA, 18 bp, Mus musculus
  source 1..18: mol_type = genomic DNA
agcagcagcagcagcagc

SEQ ID NO: 5 - DNA, 18 bp, Mus musculus
  source 1..18: mol_type = genomic DNA
gcagcagcagcagcagca

SEQ ID NO: 6 - RNA, 20 nt, Mus musculus
  source 1..20: mol_type = genomic RNA
caggcaggcaggcaggcagg

SEQ ID NO: 7 - DNA, 20 bp, Mus musculus
  source 1..20: mol_type = genomic DNA
aggcaggcaggcaggcaggc

SEQ ID NO: 8 - DNA, 20 bp, Mus musculus
  source 1..20: mol_type = genomic DNA
ggcaggcaggcaggcaggca

SEQ ID NO: 9 - DNA, 20 bp, Mus musculus
  source 1..20: mol_type = genomic DNA
gcaggcaggcaggcaggcag

SEQ ID NO: 10 - DNA, 18 bp, Mus musculus
  source 1..18: mol_type = genomic DNA
ccccggccccggccccgg

SEQ ID NO: 11 - DNA, 18 bp, Mus musculus
  source 1..18: mol_type = genomic DNA
ggggccggggccggggcc

SEQ ID NO: 12 - DNA, 24 bp, Mus musculus
  source 1..24: mol_type = genomic DNA
caggcaggcaggcaggcaggcagg

SEQ ID NO: 13 - DNA, 24 bp, Mus musculus
  source 1..24: mol_type = genomic DNA
cctgcctgcctgcctgcctgcctg

SEQ ID NO: 14 - DNA, 960 bp, Mus musculus
  source 1..960: mol_type = genomic DNA
cctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctg

SEQ ID NO: 15 - RNA, 20 nt, Mus musculus
  source 1..20: mol_type = genomic RNA
cctgcctgcctgcctgcctg

SEQ ID NO: 16 - DNA, 1440 bp, Mus musculus
  source 1..1440: mol_type = genomic DNA
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 17 - DNA, 315 bp, Mus musculus
  source 1..315: mol_type = genomic DNA
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 18 - DNA, 548 bp, Mus musculus
  source 1..548: mol_type = genomic DNA
cctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctg

SEQ ID NO: 19 - RNA, 960 nt, Mus musculus
  source 1..960: mol_type = genomic RNA
cctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctgcctg

SEQ ID NO: 20 - DNA, 720 bp, Mus musculus
  source 1..720: mol_type = genomic DNA
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 21 - DNA, 2880 bp, Mus musculus
  source 1..2880: mol_type = genomic DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 22 - DNA, 960 bp, Mus musculus
  source 1..960: mol_type = genomic DNA
caggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcaggcagg

SEQ ID NO: 23 - DNA, 2880 bp, Mus musculus
  source 1..2880: mol_type = genomic DNA
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 24 - RNA, 2880 nt, Mus musculus
  source 1..2880: mol_type = genomic RNA
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 25 - DNA, 20 bp, Mus musculus
  source 1..20: mol_type = genomic DNA
cctgcctgcctgcctgcctg

SEQ ID NO: 26 - RNA, 1440 nt, Mus musculus
  source 1..1440: mol_type = genomic RNA
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 27 - DNA, 85 bp, Mus musculus
  source 1..85: mol_type = genomic DNA
tcgacctgcctgcctgcctgcctgcctgcctgcctgcctgcctgcctgcctgcctgcctgcctgcctgcctgcctgcctgcctgc

SEQ ID NO: 28 - DNA, 65 bp, Mus musculus
  source 1..65: mol_type = genomic DNA
tcgagcagcagcagcagcagcagcagcagcagcagcagcagcagcagcagcagcagcagcagcag

SEQ ID NO: 29 - DNA, 450 bp, Mus musculus
  source 1..450: mol_type = genomic DNA
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 30 - DNA, 19 bp, Mus musculus
  source 1..19: mol_type = genomic DNA
gcagcagcagcagcagcag

SEQ ID NO: 31 - DNA, 19 bp, Mus musculus
  source 1..19: mol_type = genomic DNA
gagcagcagcagcagcagc

SEQ ID NO: 32 - DNA, 19 bp, Mus musculus
  source 1..19: mol_type = genomic DNA
ggcagcagcagcagcagca

SEQ ID NO: 33 - DNA, 26 bp, Mus musculus
  source 1..26: mol_type = genomic DNA
agcagcagcagcagcagcagcagcag

SEQ ID NO: 34 - DNA, 15 bp, Mus musculus
  source 1..15: mol_type = genomic DNA
cagcagcagcagcag

SEQ ID NO: 35 - DNA, 15 bp, Mus musculus
  source 1..15: mol_type = genomic DNA
ctgctgctgctgctg

SEQ ID NO: 36 - DNA, 20 bp, Mus musculus
  source 1..20: mol_type = genomic DNA
agccagccagccagccagcc

SEQ ID NO: 37 - DNA, 15 bp, Mus musculus
  source 1..15: mol_type = genomic DNA
ctgctgctgctgctg

SEQ ID NO: 38 - DNA, 20 bp, Mus musculus
  source 1..20: mol_type = genomic DNA
agccagccagccagccagcc

(19) United States
(12) Patent Application Publication
(10) Pub. No.: US 2025/0146018 A1
(43) Pub. Date: May 8, 2025

(54) COMPOSITIONS AND METHODS FOR IMPEDING TRANSCRIPTION OF EXPANDED MICROSATELLITE REPEATS

(71) Applicant: University of Florida Research Foundation, Incorporated, Gainesville, FL (US)

(72) Inventors: Eric Tzy-Shi Wang, Gainesville, FL (US); Tanvi Saxena, Gainesville, FL (US); Belinda Pinto, Gainesville, FL (US); John Andrew Berglund, Gainesville, FL (US)

(73) Assignee: University of Florida Research Foundation, Incorporated, Gainesville, FL (US)

(21) Appl. No.: 18/952,458

(22) Filed: Nov. 19, 2024

Related U.S. Application Data
(62) Division of application No. 16/346,163, filed on Apr. 30, 2019, now Pat. No. 12,180,499.
(60) Provisional application No. 62/415,353, filed on Oct. 31, 2016.

(51) Int. Cl. C12N 15/86; A61K 31/704; A61K 38/12; A61P 21/00; C12N 9/22; C12N 15/11

(52) U.S. Cl. CPC C12N 15/86; A61K 31/704; A61K 38/12; A61P 21/00; C12N 9/22; C12N 15/11; C12N 2310/20; C12N 2750/14123; C12N 2750/14143; C12N 2750/14171; C12N 2800/80

(57) ABSTRACT

Disclosed herein are compositions and methods for treating diseases characterized by expanded microsatellite repeats by impeding or inhibiting transcription of expanded microsatellite repeats.

### CROSS-REFERENCE TO RELATED APPLICATIONS

This Application is a Divisional of U.S. application Ser. No. 16/346,163, filed Apr. 30, 2019, entitled “COMPOSITIONS AND METHODS FOR IMPEDING TRANSCRIPTION OF EXPANDED MICROSATELLITE REPEATS”, which is a national stage filing under 35 U.S.C. § 371 of International Patent Application Serial No. PCT/US2017/059324, filed Oct. 31, 2017, entitled “COMPOSITIONS AND METHODS FOR IMPEDING TRANSCRIPTION OF EXPANDED MICROSATELLITE REPEATS”, which claims the benefit under 35 USC § 119(e) of U.S. Application Ser. No. 62/415,353, filed Oct. 31, 2016, entitled “COMPOSITIONS AND METHODS FOR IMPEDING TRANSCRIPTION OF EXPANDED MICROSATELLITE REPEATS”. The entire contents of these applications are incorporated herein by reference in their entirety.

### STATEMENT REGARDING FEDERALLY SPONSORED RESEARCH OR DEVELOPMENT

This invention was made with government support under Grant No. DP5 OD017865 awarded by the National Institutes of Health. The government has certain rights in the invention.

### REFERENCE TO AN ELECTRONIC SEQUENCE LISTING

The contents of the electronic sequence listing (U119670110US02-SEQ-GJM.xml; Size: 50,644 bytes; and Date of Creation: Nov. 18, 2024) is herein incorporated by reference in its entirety.

### BACKGROUND OF THE INVENTION

Microsatellite expansion diseases are a class of genetically inherited conditions associated with destabilization and expansion of short repetitive sequences in the genome, which cause pathogenic effects via multiple mechanisms including epigenetic silencing, RNA gain of function, and/or protein gain of function. These diseases, including myotonic dystrophy Types 1 and 2 (DM1, DM2), Fuchs' endothelial corneal dystrophy (FECD), Huntington Disease (HD), C9orf72-ALS/FTD (C9ALS/FTD) and spinocerebellar ataxias (SCAs), are often multi-systemic and can affect the central nervous system, muscle, and the heart. Somatic instability causes repeat expansion throughout the lifetime of an individual, with the most dramatic expansions reaching thousands of nucleotides in length in posmitotic tissues. Cellular toxicity in these diseases occurs partly due to transcription of the expanded repeat tract. For example, in DM1 and DM2 expanded CUG or CCUG repeat RNAs, respectively, sequester Muscleblind-like (MBNL) proteins from their endogenous RNA targets leading to aberrant splicing patterns and altered RNA stability/localization, among other effects. In C9ALS/FTD, expanded G₄C₂ and C₄G₂ repeat RNAs sequester RNA binding proteins, as well as undergo RAN translation to produce toxic dipeptide polymers

ALS is a devastating neurodegenerative disease that is characterized by both upper and lower motor neuron dysfunction, leading to paralysis, respiratory failure, and eventual death. Frontotemporal dementia (FTD) is a type of early onset dementia characterized by degeneration of the frontal and anterior temporal lobes, leading to cognitive deficits, as well as behavior and language abnormalities. The most common genetic cause of ALS and FTD is a G₄C₂ hexanucleotide repeat expansion (HRE) in the first intron of C9ORF72. Various disease mechanisms have been proposed to explain disease pathogenesis, including 1) the production of RNA species from sense and anti-sense transcripts containing HREs, which may sequester RNA binding proteins, compromising their functions, as well as 2) dipeptide repeats translated from those RNAs through a non-canonical mechanism that does not require an ATG start codon. A third potential disease mechanism that has been proposed is loss of function of the C9ORF72 transcript, due to decreased levels of the mature mRNA from the expanded allele; however, various lines of evidence suggest that haploinsufficiency does not play a significant role in disease pathology.

A number of approaches have been taken to reduce the abundance of sense and anti-sense transcripts in the context of C9ORF72/ALS/FTD. Antisense oligonucleotides targeting each transcript can reduce RNA foci as well as dipeptide synthesis, resulting in rescue of neuronal phenotypes in cell culture as well as behavioral deficits in mouse models of ALS. More recently, depletion of SUPT4H1 and/or SUPT5H in c9ALS fibroblasts has been demonstrated to reduce transcription of both sense and anti-sense HRE transcripts, as well as poly (GP) dipeptide repeats (DPRs). These genes act as co-factors for RNA polymerase II and were initially shown to be required for transcription of long repeats in the context of Huntington's disease.

Various approaches have been taken to silence toxic RNA or protein in microsatellite expansion diseases, including antisense oligonucleotides, small RNAs, and small molecules. Perturbation to co-factors of RNA polymerase II (RNA Pol II) reduces transcription through expanded repeats in HD and C9ALS/FTD models, and treatment with Actinomycin D at nanomolar doses preferentially impedes transcription of CTG repeats in DM models. A hypothesis is that efficiency of transcription through expanded repeats is decreased relative to non-repetitive sequences. This provides a therapeutic window through which to impede transcription of these sequences in a repeat length-dependent manner, resulting in premature termination and nascent transcript turnover.

A deactivated version of the Cas9 enzyme (dCas), of the clustered regularly interspaced short palindromic repeats (CRISPR) system, can be used to impair transcription of specific loci, as well as visualize, tether, and/or isolate DNA in a sequence-specific manner. In prokaryotes, dCas9 efficiently inhibits transcriptional initiation and elongation when bound to gene bodies or promoters. In eukaryotes, dCas9 inhibits transcriptional initiation when fused to an inhibitory domain and targeted near the transcription start site (TSS). However, elongation inhibition by targeting dCas9 alone to the gene body (>1 kb from TSS) has been largely ineffective, even when recruiting dCas9 to >90 possible sites.

### SUMMARY OF THE INVENTION

Compositions and methods for treating diseases characterized by expanded microsatellite repeats are disclosed. Disclosed herein are compositions and methods for reducing production of RNA and proteins arising from expanded microsatellite repeats associated with diseases including multiple human diseases, including myotonic dystrophy, Fuchs' endothelial corneal dystrophy, and C9orf72-ALS/FTD.

Systemic delivery of dCas9/gRNA by adeno-associated virus led to reductions in pathological RNA foci, rescue of chloride channel 1 protein expression, and decreased myotonia. These observations suggest that transcription of microsatellite repeat-containing RNAs is more sensitive to perturbation than transcription of other RNAs, indicating potentially viable strategies for therapeutic intervention.

An aspect of an embodiment of the methods includes administering an effective amount of deactivated S. pyogenes Cas9 or deactivated S. aureus Cas9 to a subject in need thereof. The deactivated Cas9 (dCas9) impedes or inhibits transcription of expanded microsatellite repeats using guide RNAs that directly target the microsatellite repeat tract. A repeat length-, proto-spacer adjacent motif (PAM)-, and strand-dependent reduction of repeat-containing RNAs is achieved upon targeting dCas9 directly to repeat sequences; targeting the non-template strand is preferred. Aberrant splicing patterns are rescued in DM1 cells, and production of repeat associated non-ATG (RAN) peptides characteristic of DM1, DM2, and C9orf72-ALS/FTD cells is drastically decreased.

An aspect of an embodiment of the methods includes administering an effective amount of a small molecule therapeutic that impedes or inhibits transcription of expanded microsatellite repeats to a subject in need thereof. Small molecule therapeutics that impede or inhibit transcription of expanded microsatellite repeats include, but are not limited to, actinomycin D, echinomycin and mythramycin A.

Diseases characterized by expanded microsatellite repeats include myotonic dystrophy type 1, myotonic dystrophy type 2, C9ALS/FTD, spinocerebellar ataxias, Fuch's endothelial corneal dystrophy and other neurological disorders.

An aspect of an embodiment of the compositions of matter that impedes or inhibits transcription of expanded microsatellite repeats includes an AAV vector, AAV virion or AAV viral particle that contains dCas9 and gRNA.

Disclosed herein are: 1) application of CRISPRi to block transcription of HREs in vitro and in vivo, 2) identification of small molecules that block sense and/or antisense transcription of HRE-containing C9ORF72, and 3) characterization of phenotypic changes in C9ALS mice before and after transcriptional blockade.

### BRIEF DESCRIPTION OF THE DRAWINGS

FIG. 1A is a depiction of the strategy of the methods of the invention using deactivated Cas9 to block transcription of HREs. The large number of dCas9/guide RNA complexes bound to DNA can impede transcription in a strand-specific manner.

FIG. 1B is a diagram showing identifying small molecules that impede HRE transcription, and testing CRISPRi and small molecules in a mouse model of ALS.

FIGS. 2A-2G are graphs and photographs showing that dCas9 impedes transcription of expanded repeats. FIG. 2A is a schematic of gRNA/DNA hybridization for 3 different gRNAs targeting CTG repeats. FIG. 2B is a graph showing expression of CUG repeat-containing RNA in the presence of dCas9 and gRNAs targeting repeats. Length-dependent reduction in expression is achieved with the (CAG)₆ gRNA. FIG. 2C is a bar graph showing expression of repeat relative to control. Plasmids encoding 960 CTG or CAG repeats were assayed for expression in the presence of (CAG)₆ gRNA or (CTG)₆ gRNA. The CAG PAM suppresses more effectively than CTG, and targeting the non-template strand is more effective than targeting the template strand. FIG. 2D is a bar graph showing that DM2 CCTG repeats can be effectively suppressed by guide RNAs encoding CAGG repeats. FIGS. 2E-2G are photographs of Western blots showing that dCas9 and gRNAs suppress RAN translation of CTG (FIG. 2E), CCTG (FIG. 2F) and G₄C₂ (FIG. 2G) repeats, as assayed using protein tags in frame with RAN peptides.

FIGS. 3A-3G are graphs and photographs showing that small molecules impede transcription through expanded CTG/CCTG repeats. FIG. 3A is a graph showing that three different molecules, actinomycin D (ActD), echinomycin (Echino) and mythramycin A (MTM), impede transcription of CTG240 relative to GAPDH in a concentration-dependent manner. FIG. 3B is a graph showing that ActD and Echinomycin inhibit transcription of CTG repeats in a repeat length-dependent manner. FIG. 3C is a graph showing that the three different molecules impede transcription of CCTG240 relative to GAPDH in a concentration-dependent manner. FIGS. 3D-3G show RNA foci appear in DM2 lymphoblastoid cells (LCL) but not non-DM2 LCLs, and are reduced when treated with ActD or Echinomycin.

FIGS. 4A-4H are graphs and photographs showing features of C9-BAC mice. FIG. 4A is a graph showing reduced survival, FIG. 4B is a bar graph showing that 82% are phenotypic at 1 year, FIG. 4C is a photograph showing paralysis, FIG. 4D is photographs showing neuromuscular junction denervation, FIG. 4E is photographs showing neurodegeneration of motor cortex, FIG. 4F is photographs and a bar graph showing spinal motor neuron loss, FIG. 4G is photographs showing hippocampal neuron loss, and FIG. 4H are plots and a bar graph showing open-field anxiety-like cage behavior.

FIGS. 5A-5E are diagrams and graphs showing that deactivated Cas9 impedes transcription of expanded microsatellite repeats in length, PAM, and strand-dependent manner. FIG. 5A shows a proposed model for how recruitment of multiple dCas9/gRNA complexes to expanded microsatellite repeats impedes transcription of mRNA. FIG. 5B is a schematic of proposed gRNA hybridization to microsatellite repeat tract. Abundance of CUG repeat-containing RNA in the presence of dCas9/gRNAs targeting the repeat tract, relative to RNA containing 0 repeats in the presence of control gRNA. FIG. 5C is a bar graph showing the abundance of CCUG repeat-containing RNA in the presence of dCas9/gRNAs, relative to RNA with 0 repeats in the presence of control gRNA. FIG. 5D is a bar graph showing the abundance of RNAs containing 960 CUG repeats or 960 CAG repeats in the presence of (CAG)₆ gRNA or (CTG)6 gRNA. Comparisons are drawn between PAM sequence, as well as targeted strand (non-template, NT vs. template, T). FIG. 5E is a bar graph showing the abundance of RNAs containing 240 CCUG repeats or 240 CAGG repeats, in the presence of (CAGG)₆ gRNA or (CCTG)₆ gRNA, as in (FIG. 5D). Error bars are plotted as the mean+SD values of 4experiments.

FIGS. 6A-6F are graphs and photographs showing transcriptional inhibition by CRISPRi rescues molecular and cellular phenotypes in cell culture models of DM and other microsatellite expansion repeats. FIG. 6A is a bar graph showing the percentage of cells containing RNA foci in the presence of dCas9 and control or (CAG)₆ gRNA (left panel), and a graph showing the cumulative distribution of number of foci per cell in HeLa cells transfected with 480 CUG repeats (right panel). FIG. 6B is a bar graph showing the inclusion level of MBNL1 exon 5 transcribed from a minigene reporter, in the presence and absence of 480 CUG repeats, dCas9, and gRNAs that target the template or non-template strand of the repeat-containing plasmid. FIG. 6C is a graph showing the inclusion level of MBNL1 exon 5 in the presence and absence of (CAG)₆ gRNA, (CTG)₆ gRNA, or no gRNA. FIGS. 6D-6F are photographs of Western blots showing that dCas9 and repeat-targeting gRNAs suppress RAN translation of CTG (FIG. 6D), CCTG (FIG. 6E), and G₄C₂ (FIG. 6F) repeats, as assayed using protein tags in frame with RAN peptides. RAN products are indicated using brackets.

FIGS. 7A-7F are diagrams and graphs showing that deactivated SpCas9 impedes transcription of expanded microsatellite repeats in a length-, PAM-, and strand-dependent manner. FIG. 7A is a diagram of a proposed model for how recruitment of dCas9/gRNA complexes to expanded microsatellite repeats impedes transcription by RNA Pol II FIG. 7B is a schematic (top) and graph (bottom) of gRNAs used to target transcription of CTG repeats (top). Abundance of CUG repeat RNA in the presence of dCas9/gRNAs targeting the repeat tracts of various lengths in HeLa cells, relative to the same RNA species with zero repeats (bottom). Error bars show standard deviation. FIG. 7C is a graph showing relative dCas9 ChIP signal across all repeat lengths versus percent RNA remaining following (CAG)₆ gRNA relative to control gRNA treatment. dCas9 ChIP signal is computed as dCas9 IP divided by input chromatin with (CAG)₆ gRNA treatment, divided by dCas9 IP divided by input chromatin with control gRNA treatment. Relative abundance of repeat-containing loci and RNAs was assessed by deep sequencing of the barcodes. Error bars show standard error of the mean. FIG. 7D is a bar graph showing abundance of CCUG repeat RNA in the presence of dCas9 and gRNAs targeting the (CCTG)₂₄₀ repeat tract in HeLa cells, relative to the same RNA species with zero repeats. FIG. 7E is a bar graph showing abundance of RNAs containing 960 CUG or CAG repeats in the presence of dCas9 and (CUG)₆ or (CAG)₆ gRNAs in HeLa cells, relative to RNA species with zero repeats. Arrows denote comparisons relevant for assessing PAM-or strand-dependent effects on efficacy. FIG. 7F is a bar graph showing abundance of RNAs containing 240 CCUG or CAGG repeats in the presence of dCas9 and (CCUG)₅ or (CAGG)₅ gRNAs in HeLa cells, relative to RNA species with zero repeats. Arrows denote comparisons relevant for assessing PAM-or strand-dependent effects on efficacy. (All significance tests are by two-tailed T-test, **p<0.0005, *p<0.005)

FIGS. 8A-8G are photographs and graphs showing that transcriptional inhibition rescues molecular and cellular phenotypes in cell culture models of DM1, DM2, and C9ORF72/ALS/FTD. FIG. 8A is a series of photographs showing representative FISH-IF images of HeLa cells transfected with plasmids expressing (CTG)₄₈₀ repeats, HA-dCas9 and control or (CAG)₆ gRNAs. RNA foci are shown in red, dCas9 in green and DNA (DAPI) in blue. Scale bar: 10 μm FIG. 8B is a bar graph (left panel) and a line graph (right panel) showing the quantitation of HA-positive cells showing nuclear RNA foci (left panel) and the cumulative density function of HA-positive cells with a given number of RNA foci (right panel), in the presence of dCas9 and control or (CAG)₆ gRNAs (Control: 139 cells, (CAG)₆: 111 cells, Kolmogorov-Smirnov test, *p<0.005). FIG. 8C is a bar graph of of MBNL1 exon 5 expressed in a minigene context in HeLa cells, in the presence of combinations of plasmids encoding (CTG)₄₈₀ repeats, dCas9, and (CAG)₆ or (CUG)₆ gRNAs. N>3 for all conditions (two-tailed T-test, *p<0.005) FIG. 8D is a graph of Ψ of the MBNL1 exon 5 minigene in HeLas, in the presence of plasmids encoding 0, 12, 40, 240, 480, or 960 CTG repeats, as well as dCas9 and (CAG)₆ or (CUG)₆ gRNAs. N>3 for all conditions. Error bars are too small to be visible. FIG. 8E is a photograph of a Western blot to detect FLAG-tagged poly-Ala RAN peptides expressed from DM1 CTG repeats. Cells were transfected with various combinations of plasmids encoding no or (CTG)₁₀₅ repeats with combinations of dCas9 and control, (CAG)₆ or (CUG)₆ gRNAs. The peptides migrate as a 75 and 150 kD smear. The asterisk indicates a cross-reacting protein produced by the dSpCas9 plasmid. FIG. 8F is a photograph of a Western blot against the HA-tagged LPAC RAN protein expressed from DM2 CCTG repeats. Cells were transfected with various combinations of plasmids encoding no or (CCTG)₁₃₇ repeats with combinations of dCas9 and control, (CAGG)₅, (AGGC)₅, (GCAG)₅ or (CCUG)₅, gRNAs. The peptide migrates at 20 kD. FIG. 8G is a photograph of a Western blot against the FLAG-tagged poly-GlyPro RAN protein expressed from ALS G₄C₂ repeats. Cells were transfected with various combinations of plasmids encoding no or (G₄C₂)₁₂₀ repeats with combinations of dCas9 and control, (C₄G₂)₃ or (G₄C₂)₃ gRNAs. The peptides migrate as a 75 and 120 kD smear. The asterisk indicates a cross-reacting protein produced by the dSpCas9 plasmid, β-Actin serves as the loading control and N=3 for FIGS. 8E, 8F and 8G (representative blot shown).

FIGS. 9A-9F are photographs and graphs showing that AAV-dSaCas9 rescues molecular and cellular phenotypes in human DM1 myoblasts. FIG. 9A is a series of photographs of representative FISH-IF images of DM1 myoblasts infected with AAV-dSaCas9-control or (CAG)₆ gRNAs. RNA foci are shown in red, dCas9 in green, and DAPI in blue. Scale bar: 10 μm FIG. 9B is a graph showing the probability density function of cells with a given number of RNA foci, in the presence of control or (CAG)₆ gRNAs (Kolmogorov-Smirnov test for statistical significance). FIG. 9C is a graph of MISO estimates of MBNL1 exon 5 Ψ in 11 unaffected and 44 DM1 TA biopsies plotted in order of [MBNL]_{inferred} as previously described. A sigmoid curve was fit to these points and shown (dashed line) with 90% confidence intervals (gray shading). MISO estimates of MBNL1 exon 5 Ψ values in unaffected and DM1 primary myoblast lines (2 and 4 replicates, respectively) are shown in blue and red lines, respectively, with ranges also shaded. FIG. 9D is a scatter plot for splicing events regulated in both TA biopsies and the human myoblast lines described in (FIG. 9C). 115 splicing events were selected from TA biopsies based on best sigmoid fits with [MBNL]_{inferred} as in (FIG. 9C). The x-axis of the scatter plot is the mean Ψ in the most severely affected biopsies (<0.33 [MBNL]_{inferred}) minus the mean Ψ across unaffected individuals. The y-axis of the scatter plot is the mean Ψ in the DM1 myoblast minus the mean 4 in the unaffected myoblast line. Labeled points have ΔΨ>0.3 in both conditions, a sigmoid fit<1.3, and a y-axis monotonicity score>1 (see Examples). Pearson correlation is listed. FIG. 9E is a bar graph of MBNL1 exon 5 Ψ, assessed by RT-PCR in unaffected myoblasts and DM1 myoblasts infected with AAV-dSaCas9-control or (CAG)₆ gRNAs. FIG. 9F is a scatter plot illustrating changes in Ψ in response to AAV-dSaCas9 (CAG)₆ gRNA, for splicing events exhibiting concordant behavior in (FIG. 9D). The x-axis is as in (FIG. 9D), but the y-axis is ΔΨ in DM1 myoblasts, AAV-dSaCas9-control gRNA treatment minus AAV-dSaCas9-(CAG)₆ gRNA treatment. Labeled points have ΔΨ>0.1 in both conditions and a y-axis Bayes Factor>5 (see Examples). Pearson correlation is listed.

FIGS. 10A-10C are photographs and graphs showing that AAV-dSaCas9-(CAG)6 reduces RNA foci in HSA^{LR} muscle fibers. FIG. 10A is a series of photographs of FISH to detect nuclear RNA foci (magenta) in myonuclei of HSA^{LR} EDL muscle fibers that were untreated (top), infected with AAV-dSaCas9-control gRNA (middle) or (CAG)₆ gRNA (bottom). A representative fiber for each condition is shown (Scale bar: 100 μm). Insets from each fiber (white boxes) are on the right (Scale bar: 50 μm). DAPI is in cyan. FIG. 10B is a graph showing the percentage of myonuclei per fiber showing RNA foci quantitated across all 3 conditions with 5-6 fibers per condition, 400-500 nuclei per fiber (two-tailed T-test, **p<0.0005). FIG. 10C is a graph of the probability density function of intensity of FISH signal in myonuclei from untreated fibers (red), fibers infected with AAV-dSaCas9-control gRNA (orange) or AAV-dSaCas9-(CAG)₆ gRNA (yellow). A Kolmogorov-Smirnov test was performed for statistical significance.

FIGS. 11A-11D are photographs and graphs showing that AAV6-dSaCas9-(CAG)6 rescues muscle phenotypes in HSA^{LR} mice. FIG. 11A is a graph of percent needle insertions showing a myotonic run in wild type FVB, MBNL1^{-/-}, HSA^{LR} (black) and HSA^{LR} treated with AAV6-dSaCas9-control gRNA (orange) or (CAG)₆ gRNA (yellow). Each point represents a muscle from a single animal, but in some animals, two muscles were assayed. (p<0.027 for AAV6-dSaCas9-control versus AAV6-dSaCas9-CAG₆ treated animals, two-tailed T-test) FIG. 11B is photographs of IF against HA-dSaCas9 (green) in an EDL fiber from a mouse treated with AAV6-dSaCas9-(CAG)₆ gRNA. DAPI is in blue Scale bar: 50 μm. FIG. 11C is a bar graph showing quantitation of EDL fibers with complete, partial, or no CUG RNA foci upon treatment with AAV6-dSaCas9-control or (CAG)₆ gRNA. Myotonia levels and numbers of fibers analyzed are also listed. FIG. 11D is a series of photographs showing IF against Clcn1 in TA muscle sections from FVB, HSA^{LR}, and HSA^{LR} treated with AAV-dSaCas9-control or (CAG)₆ gRNA. DAPI is shown in blue. Scale bar: 50 μm.

FIG. 12 is a diagram of a model for microsatellite repeat expansion targeting by dCas9. dCas9 binding to DNA impedes transcription of long, expanded microsatellite repeats. In the context of shorter repeats, fewer copies of DNA-bound dCas9 may be insufficient to fully inhibit elongation by RNA Pol II, permitting production of RNA that can also be targeted by dCas9.

FIGS. 13A-13C are diagrams showing Measurement of Barcoded Transcripts by Amplicon Sequencing (MBTA-Seq). Related to FIG. 7. FIG. 13A is a schematic illustrating barcoded plasmids containing various repeats of different lengths (pink box) followed by unique 8-nt barcodes (blue box). The table lists the unique barcodes for each repeat type. FIG. 13B is a diagram of the MBTA-Seq protocol followed to measure levels of transcripts expressed from barcoded plasmids. FIG. 13C is a schematic describing the primers used to PCR amplify the barcode region and generate amplified fragments with appropriate sequences at the ends to facilitate transcript measurement by deep sequencing.

FIGS. 14A-14G are graphs, diagrams and photographs showing that gRNA alone without dCas9 does not significantly affect levels of expanded microsatellite repeat transcript and RAN translated products. Related to FIGS. 7-8. FIG. 14A is a graph showing abundance of CUG repeat-containing RNA in the presence of various gRNAs targeting CTG repeat tracts of various lengths, relative to abundance of RNA with no CUG repeats. FIG. 14B is a diagram of the procedure to establish stable cell line that simultaneously expresses transcripts containing 0, 12, 40, 240, 480, and 960 CTG repeats, with associated 8 nucleotide-long barcodes. Plasmids were subcloned into plasmids with Piggybac terminal repeats, which facilitate genomic integration when co-expressed with the Piggybac transposase. All repeat transcripts are driven by EF1α promoter, and plasmids also contain a PGK promoter-driven puromycin cassette. HeLa cells were transfected with all 7 plasmids, selected by puromycin, and sorted for single cells by flow cytometry. 48 clones were isolated and screened by MBTA-Seq. 2 cell lines were successfully identified to contain all 6 repeat transcripts. FIG. 14C is a bar graph showing the abundance of CCUG repeat-containing RNA in the presence of various gRNAs targeting the (CCTG)₂₄₀ repeat tract, relative to abundance of RNA with no CCUG repeats. FIG. 14D is a photograph of a Western blot analyses of the poly-Ala RAN translated product (FLAG-tagged) expressed from DM1 (CTG)₁₀₅ repeats transfected into HEK293T cells. Cells were transfected with plasmids carrying no repeats or (CTG)₁₀₅ repeats and combinations of control, (CAG)₆ or (CUG)₆ gRNAs with and without dCas9. The RAN product migrates as a smear between 75 and 150 kD. FIG. 14E is a photograph of a Western blot against the poly-LPAC RAN translated product (HA-tagged) expressed from DM2 (CCTG)₁₃₇ repeats transfected into HEK293T cells. Cells were transfected with no repeats or (CCTG)₁₃₇ repeats and combinations of control, (CAGG)₅, (AGGC)₅, (GCAG)₅ or (CCUG)₅, gRNAs with and without dCas9. The RAN translated product runs at around 20 kD. FIG. 14F is a photograph of a Western blot against the poly-GP RAN translated product (FLAG-tagged) expressed from C9orf72-ALS/FTD (G₄C₂)₁₂₀ repeats transfected into HEK293T cells. Cells were transfected with plasmids carrying no repeats or (G₄C₂)₁₂₀ repeats and combinations of control, (C₄G₂)₃ or (G₄C₂)₃ gRNAs with and without dCas9. The RAN product migrates as a smear between 75 and 120 kD. The asterisk indicates protein produced by the dSpCas9 plasmid, which cross-reacts with the anti-FLAG antibody. β-Actin serves as the loading control for FIGS. 14D, 14E and 14F. N=3 for FIGS. 14D, 14E, and 14F (representative blot shown). FIG. 14G is a graph showing that dCas9 RIP was performed to quantitate the relative abundance of various repeat-containing transcripts bound by dCas9, using the cell line described in panel (FIG. 14B). Anti-HA antibody was used to immunoprecipitate dCas9 in cells treated with control gRNA or (CAG)₆ gRNA. RNA was isolated from total lysate, or from the immunoprecipitation. The relative enrichment is plotted as a function of repeat length, e.g. (CAG)₆ gRNA IP divided by (CAG)₆ gRNA input, divided by control gRNA IP divided by control gRNA input.

FIGS. 15A-15D are graphs and photographs showing that transcriptional block of microsatellite repeat constructs by dSaCas9 in HeLa cells and dSaCas9 localization in infected DM1 primary myoblasts. Related to FIG. 9. FIG. 15A is a graph showing abundance of CUG repeat-containing RNA in the presence of control or (CAG)₆ gRNA targeting 0-960 CTG repeat tracts relative to abundance of RNA with no CUG repeats in HeLa cells FIG. 15B is a series of photographs of immunofluorescence analyses to detect expression of dSaCas9 in DM1 primary myoblasts infected with AAV-dSaCas9_{ctrl} (top) and AAV-dSaCas9_{CAG} (bottom). HA-tagged Cas9 (green) is predominantly nuclear, but also found in the cytoplasm at lower levels. Nuclei are shown in blue. FIGS. 15C and FIG. 15D are graphs of the analysis of gene expression changes between non-DM1 myoblasts treated with AAV-dSaCas9 and (CAG)₆ gRNA and those treated with AAV-dSaCas9 and control gRNA. Genes were grouped by the maximum number of contiguous CTG repeats (FIG. 15C) or CAG repeats (FIG. 15D) found in their pre-mRNA sequences. Numbers of genes found in each group are shown above. Y-axis shows fold-change in log₂ units.

FIGS. 16A-16E are photographs and graphs showing the computational analysis of foci intensity in muscle fibers, and experiments to confirm even staining across muscle sections. Related to FIGS. 10-11. FIG. 16A is a diagram used to minimize computational processing of images, regions of interest were first defined in ˜1000×1000×100 blocks. FIG. 16B is a series of photographs and a graph showing that nuclei were segmented using standard image processing techniques. Images were smoothed with a Gaussian filter, thresholded, and segmented for nuclei. FIG. 16C is a series of photographs and graphs showing that FISH signal was similarly processed. Background was subtracted, and FISH signal within nuclei was quantitated by applying the nuclei mask from (FIG. 16B). Also see Examples. FIG. 16D is a graph of percent needle insertions showing a myotonic run in wild type FVB, MBNL1^{-/-}, HSA^{LR}, (black circles) and HSA^{LR} treated with AAV9-dSaCas9 with control gRNA (orange circles) or (CAG)₆ gRNA (yellow circles). Each point represents a muscle from a single animal, but in some animals, both tibialis anterior and gastrocnemius were assayed. N=4 mice for control gRNA and N=4 mice for (CAG)₆ gRNA. FIG. 16E is photographs showing immunofluorescence performed against lamin A using an anti-lamin A antibody on mouse EDL muscle fibers, showing uniform detection of lamin A in myonuclei along the length of the fiber.

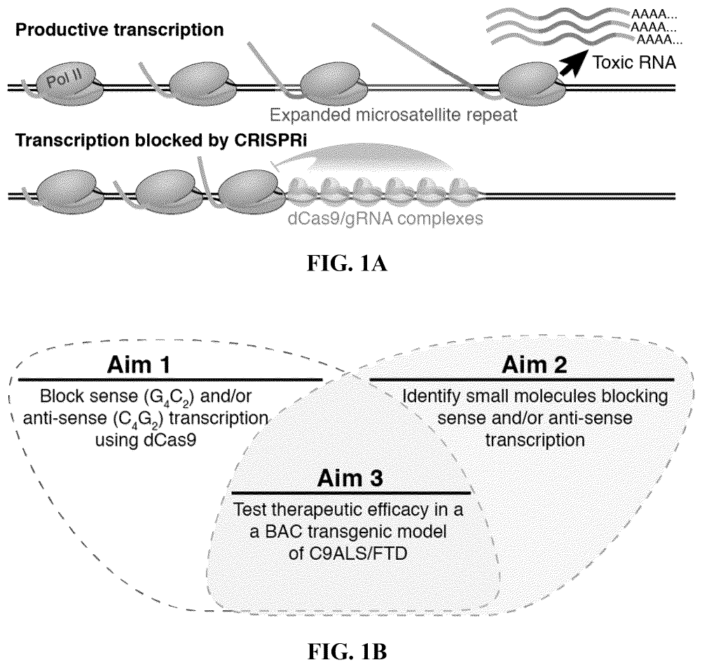

### DETAILED DESCRIPTION OF THE INVENTION

Expanded microsatellite repeats are highly sensitive to transcriptional blockade by dCas9, even in the context of elongating RNA Pol II. It was found that efficiency of inhibition follows rules similar to those observed in non-repetitive contexts, with clear dependencies on proto-spacer adjacent motif (PAM) sequence and targeted DNA strand. In addition, application of this approach to cell and animal models of disease rescues downstream pathogenic consequences. These observations describe application of the CRISPR/Cas9 system to impede transcription of expanded microsatellites in a strand-dependent manner, and demonstrate that targeting transcription in a repeat length-dependent manner is a viable therapeutic strategy for these diseases.

Amotrophic lateral sclerosis (ALS) is a devastating disease affecting approximately 1 in 25,000 individuals in the US. The most common genetic cause of ALS is a hexanucleotide G4C2 repeat expansion (HRE) found within the first intron of C9orf72. In addition to motor neuron disease, this repeat can also cause frontotemporal dementia (FTD), at times comorbid with motor neuron disease. The length of the HRE is inversely correlated with age of onset, as well as survival after disease onset. Evidence indicates that both sense (G4C2) and antisense (C4G2) RNAs are transcribed from these repeats, repeat associated non-ATG (RAN) peptides are translated from these RNAs, and that these molecules are toxic to cells. Proof of principle experiments reducing abundance of HRE-containing RNAs have rescued molecular and physiological phenotypes in cell and animal models of C9ALS/FTD, either through using antisense oligonucleotides, or by perturbing co-factors of RNA Polymerase II to elicit elongation failure across HREs.

While these studies suggest that reducing toxic RNA abundance will rescue pathology in C9ALS/FTD, a need exists to establish the relative contribution of sense vs. antisense HRE transcripts to disease, so that the appropriate molecules can be successfully targeted. In the context of therapeutic development, it is critical to characterize molecular events downstream of each transcript species so that potential benefit can be accurately measured. To date, understanding the role of individual transcripts in disease has been technically difficult due to challenges in targeting individual transcripts, and the need for an animal model that express both sense and antisense transcripts, and exhibits robust phenotypes mirroring the human disease. To address these challenges, deactivated (d)Cas9 with HRE-targeting guide RNAs are being used to inhibit transcription of expanded microsatellite repeats in a strand-specific manner (CRISPRi). A BAC-transgenic mouse model of C9ALS/FTD has been developed that recapitulates bidirectional transcription and the severe neurodegenerative phenotypes of the human disease, including loss of upper and lower motor neurons. This mouse model and other cell-based models of ALS will be used to 1) characterize the molecular pathophysiology downstream of sense and antisense repeat transcription, 2) evaluate the therapeutic impact of dCas9-mediated transcriptional blockage of one or both transcripts, and 3) identify small molecules that block sense and/or antisense transcription in cell and animal models of C9ALS/FTD.

A deactivated form of the clustered regularly interspaced short palindromic repeats (CRISPR) system has been shown to efficiently block transcription, either by tethering transcriptional inhibitory domains to eukaryotic promoters, or alone, without any additional protein cargo, in bacteria (CRISPR inhibition, CRISPRi). Expanded microsatellite repeats present significant challenges for elongating eukaryotic RNA polymerase II, and targeting deactivated Cas9 to repeats could block transcription of repeats much more efficiently than other loci lacking such long contiguous repeats. In preliminary studies, efficient blockade of expanded CTG, CCTG, and G₄C₂ transcription was demonstrated, as assayed by RNA abundance as well as RAN protein production.

CRISPRi will be used to validate transcriptional inhibition as a therapeutic approach for C9ALS, downstream consequences of blocking sense versus anti-sense transcription will be evaluated, and small molecules will be identified that can efficiently, yet selectively impede transcription of HREs. A previous limiting factor in C9ALS therapeutic studies was the availability of a mouse model recapitulating human disease phenotypes, in particular lower motor neuron phenotypes. A BAC-based model fulfilling this need has recently been developed, and it will be used to measure changes in disease pathology upon blocking sense and/or antisense transcription.

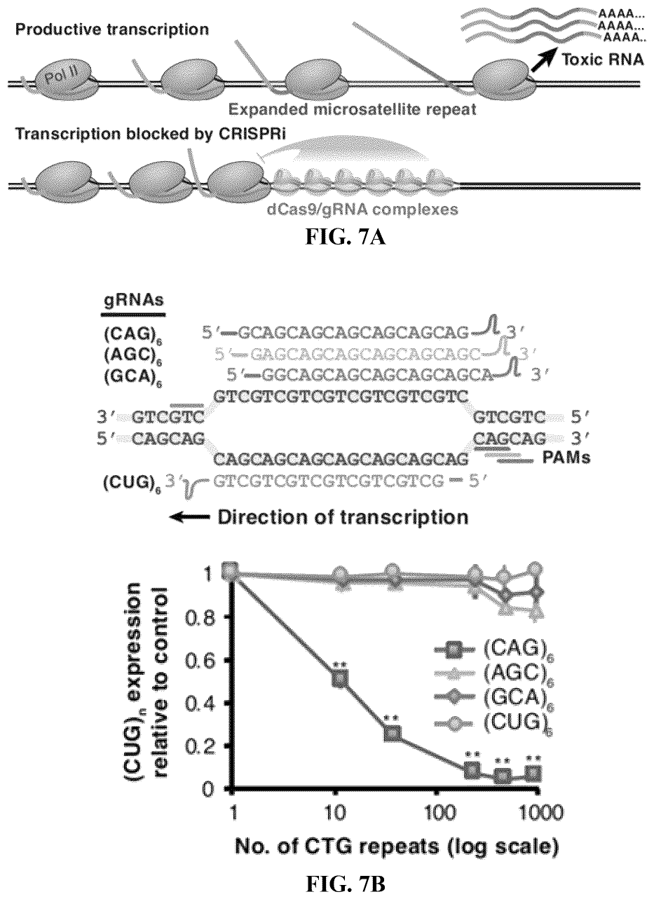

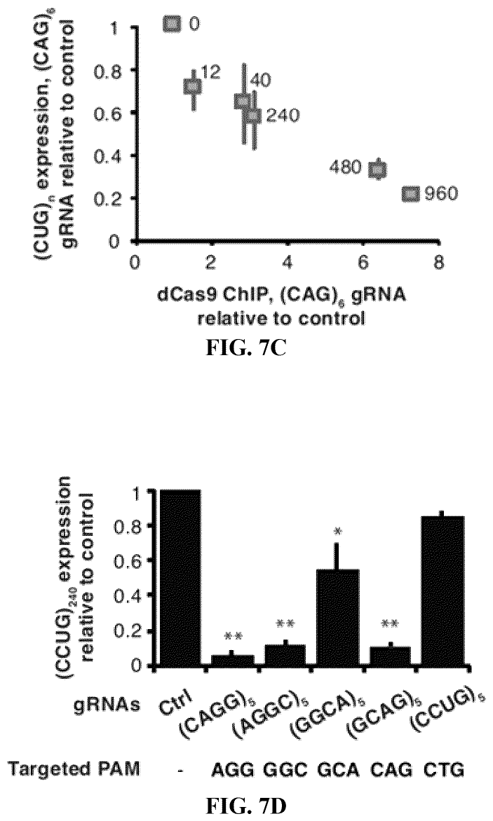

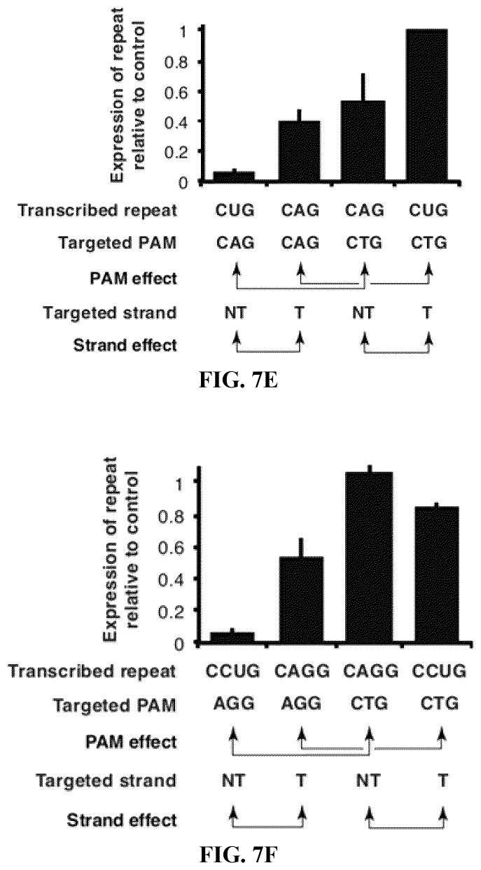

CRISPR/Cas9 has been previously applied to microsatellite expansion diseases, to remove expanded repeat tracts or to cause repeat contraction. Here a deactivated form of Cas9 was applied to these diseases. By testing disease-associated repeat sequences across multiple repeat lengths in vitro and in disease models, it was demonstrated that dCas9 can substantially reduce repeat-containing transcript abundance. Importantly, repression efficiency is proportional to repeat length, because longer repeats recruit an increased number of dCas9/gRNA complexes, leading to greater transcriptional blockade (FIG. 7B-C).

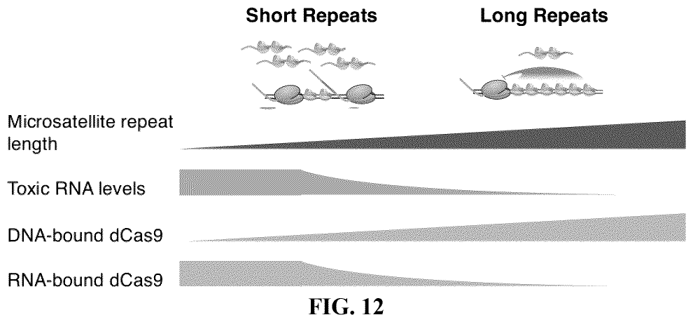

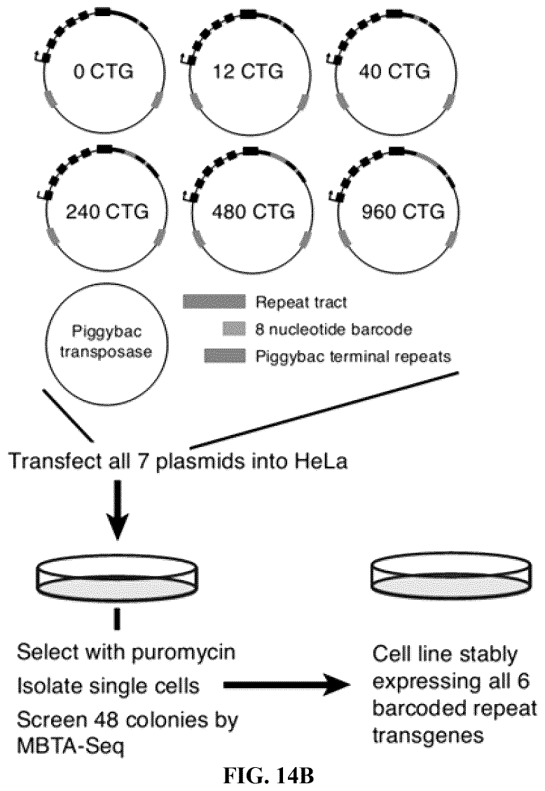

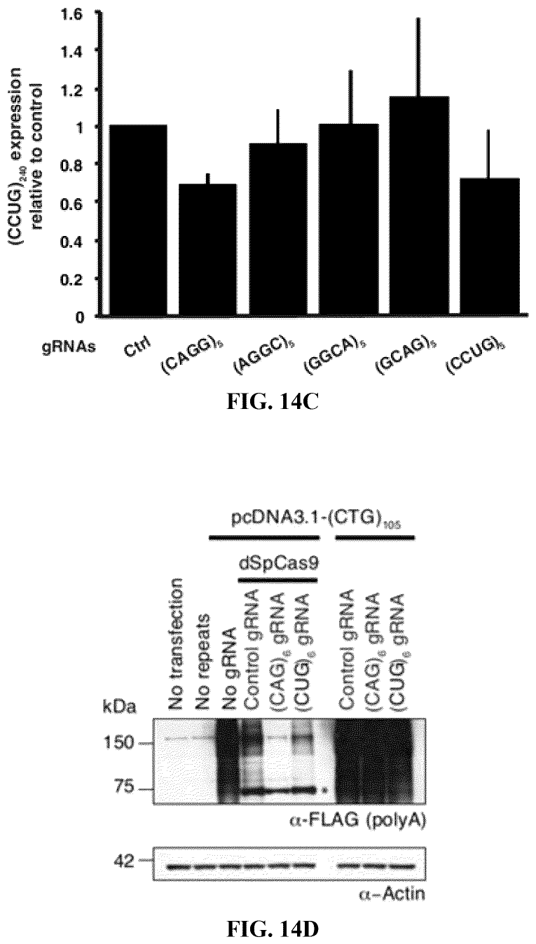

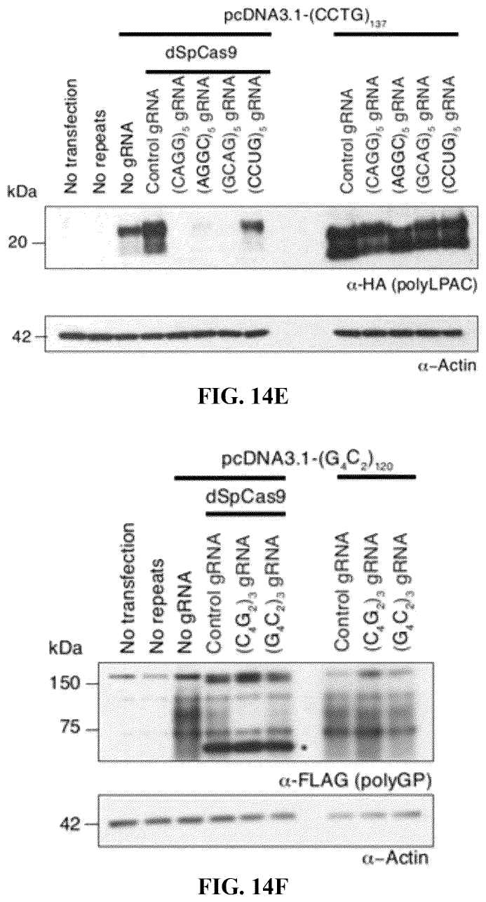

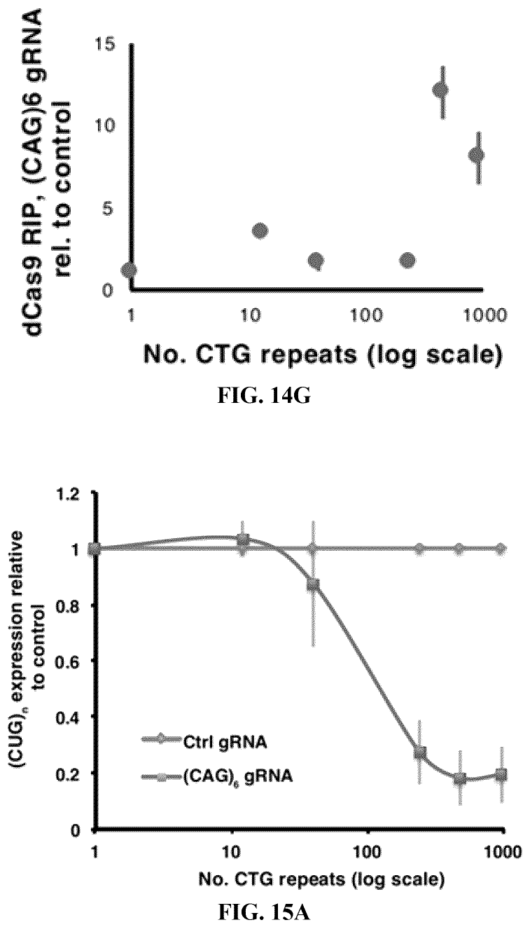

dCas9 is well established to target DNA, but has also been shown to bind repeat-containing RNA in a gRNA-dependent manner. Indeed, when dCas9 was immunoprecipitated and RNA binding was assessed, increased binding to transcripts containing longer repeats was observed (FIG. 14G). However, as repeat length increases, the amount of successfully transcribed RNA decreases, suggesting that at long repeat lengths, less RNA remains to be targeted. In the context of disease, microsatellite repeat tracts undergo dramatic somatic expansion in DM1, DM2, C9ALS/FTD, and HD, and evidence suggests that repeat lengths in tissues of symptomatic individuals reach thousands of nucleotides in length. Therefore, DNA-based targeting by dCas9 may play a primary role in driving potential therapeutic benefits in individuals with full expansions (FIG. 12). At short and intermediate repeat lengths, therapeutic benefit may derive from both DNA- and RNA-based targeting by dCas9, to not only impede transcription of repeat-containing DNA, but also modulate downstream consequences of transcribed “escaper” RNAs. Taken together, it was demonstrated that dCas9 is an effective tool to limit downstream effects of toxic RNAs.

While this approach mitigates a key safety concern of CRISPR, which is the unwanted cleavage of off-targets, it raises questions about whether long-term exposure to dCas9 can be achieved and tolerated. Effective, safe delivery in a multi-systemic fashion to sufficient numbers of post-mitotic cells will be necessary for this approach to be therapeutically viable. In DM and C9ALS/FTD, it is unclear what fraction of nuclei must be rescued in muscle or central nervous system to halt or improve disease symptoms. Here, it was observed that rescue of a subset of nuclei was sufficient to yield partial rescue of myotonia. However, myotonia can be modeled as a loss-of-function event in which Clcn1 protein is lost, and there is evidence that in other recessive muscle diseases, gene editing of a small subset of nuclei can restore sufficient protein expression across muscle fibers to yield therapeutic benefit. Conversely, mis-splicing events yielding pathogenic isoforms with dominant-negative behavior may require full elimination to mitigate deleterious consequences.

Systemically delivered AAV was used, which revealed regional variation in transduction efficiency, likely because NLS-tagged Cas9 protein remained restricted to myonuclear domains transduced by AAV episomes, and many new myonuclei are recruited to fibers throughout postnatal muscle development. Previous studies with AAV-mediated gene therapy typically evaluate expression of proteins that can spread throughout muscle fibers, precluding measurement of transduction efficiency to all myonuclei. It is possible that localized delivery would have facilitated transduction of a higher proportion of myonuclei. Nevertheless, reduction of toxic RNA repeat load in a subset of nuclei was sufficient to increase production of Clcn1 protein, with consequences for distant regions of muscle potentially naïve to dCas9. These results emphasize how differences in mechanism of action and delivery efficiency between various therapeutic approaches should significantly influence how changes in response to those therapies are interpreted, both at the molecular and phenotypic level.

In summary, the ability of dCas9 to impede transcription of microsatellite expansions defines a window in which transcriptional inhibition of expanded repeats is feasible, yet does not interfere with that of typical RNAs. This approach serves as a baseline with which to compare alternative therapeutic approaches, as well as a tool to identify mechanisms and principles by which transcriptional activity of RNA polymerase II can be modulated in a sequence-specific manner.

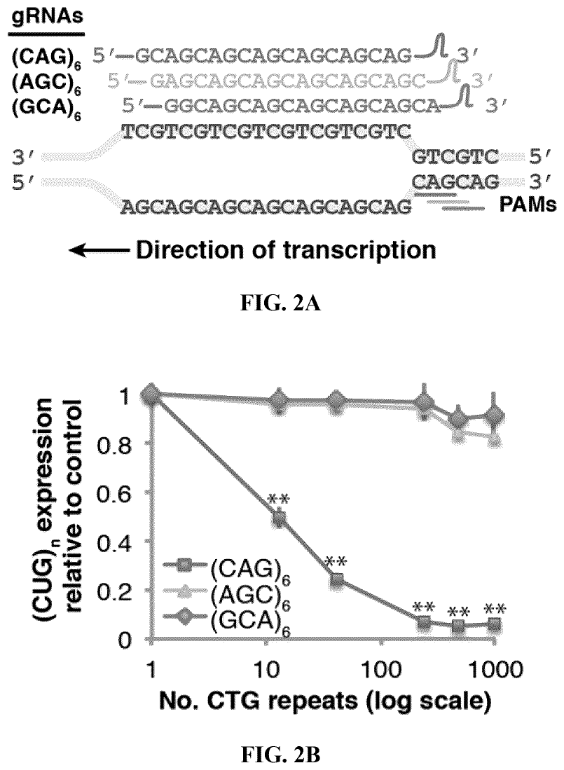

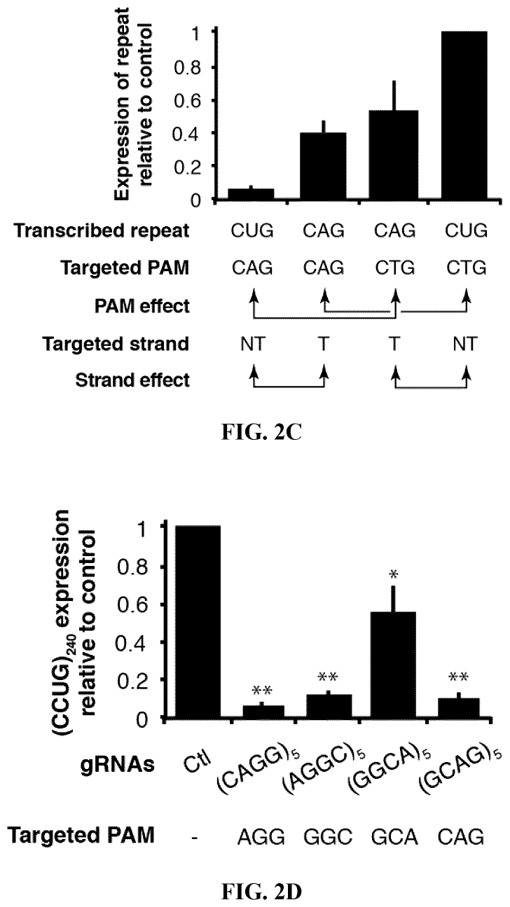

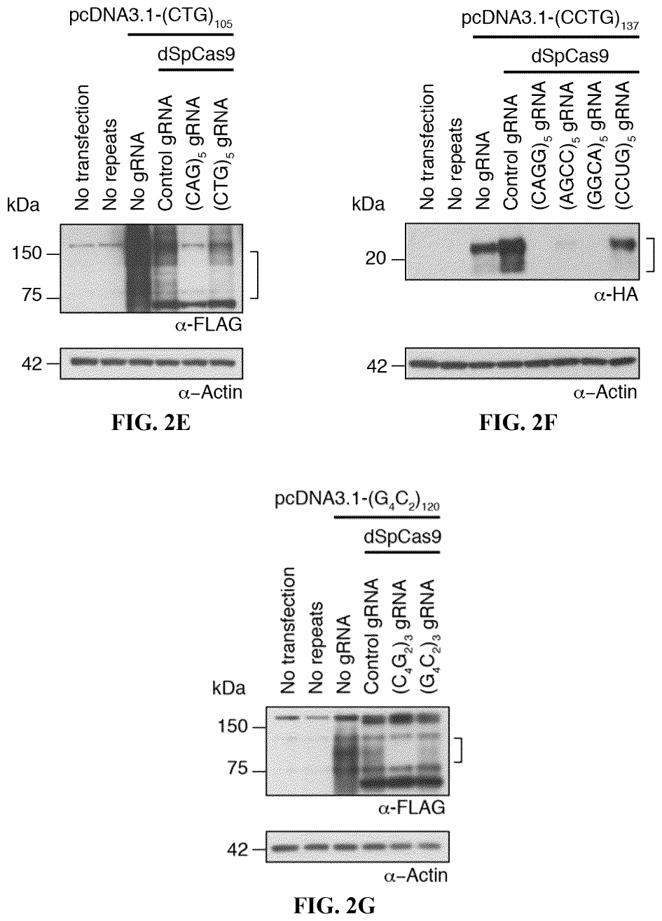

CRISPRi was used to block transcription of expanded microsatellite repeats. Deactivated Cas9 has been demonstrated to work most efficiently when fused to a transcriptional inhibitory domain (e.g., KRAB domain), and targeted by a guide RNA close to the transcriptional initiation site. However, because Pol II elongates less efficiently through expanded microsatellite repeats potentially due to alternative DNA structures, transcription could be impeded by targeting multiple copies of dCas9 to the repeat tract using a repeat-targeting guide RNA. A plasmid-based systems in cell culture was used to test this approach, and effective transcriptional blockade of expanded CTG repeats (myotonic dystrophy type 1), CCTG repeats (myotonic dystrophy type 2), and G4C2 repeats was observed. In the context of DM1 repeats, knockdown is most efficient with a protospacer-adjacent motif (PAM) of CAG, then AGC, and finally GCA, consistent with PAM efficacy (NGG>NAG>NCG>NTG)¹⁶ (FIG. 2A-B). Also, targeting of expanded CTG repeats using a (CAG)₆ gRNA is much more effective than targeting expanded CAG repeats using a (CTG)₆ gRNA (FIG. 2C), consistent with the previous observation that inhibiting Pol II via gRNA binding to the non-template strand is more effective than gRNA binding to the template strand, likely due to helicase activity of Pol II on the template strand. Similar suppression of transcription is observed with expanded CCTG/CAGG repeats (FIG. 2D). RAN translation of CTG, CCTG, and G₄C₂ repeats is also suppressed in a dCas9 and gRNA-dependent manner (FIG. 2E-G). Serendipitously, at least for Cas9 variants Streptococcus pyogenes and Staphylococcus aureus, effective targeting of both sense and antisense transcripts can be achieved because the G₄C₂ HRE sequence contains optimal PAMs on both strands. We should be able to preferentially block sense transcription using a (C₄G₂)₃ gRNA, or antisense transcription using a (G₄C₂)₃ gRNA. Indeed, RAN protein produced from transfected plasmids encoding (G₄C₂)₁₂₀ is blocked effectively by (C₄G₂)₃ gRNA, but not as effectively by (G₄C₂)₃ gRNA (FIG. 2G).

For studies with difficult-to-transfect cell types, as well as in animal tissues, S. aureus dCas9 and gRNAs have been packaged into a single adeno-associated virus (AAV), and high infectivity in human myoblasts and dissected mouse muscle fibers in culture was observed. A reduction in RNA foci was observed, and rescue of mis-splicing in DM1 was observed. In a similar approach for C9ALS/FTD, dCas9 and either a G₄C₂ repeat-targeting gRNA or a C₄G₂ repeat-targeting gRNA will be packaged in AAV. Assays will be run for reduction of RNA foci and RAN protein in C9ALS/FTD patient-derived fibroblast and lymphoblastoid cells. Then, in vivo assays will be performed in a mouse model, which exhibits both upper and lower motor neuron deficits. Then sense/antisense RNA foci will be measured, RAN protein production will be measured, other histopathological abnormalities characteristic of human C9ALS/FTD will be measure, and physiological phenotypes will be observed. The recently described viral capsids, PHP.A and PHP.B, that can efficiently transduce the central nervous system, including neurons, glia, and oligodendrocytes of the brain and spinal cord via intravenous injection will be used.

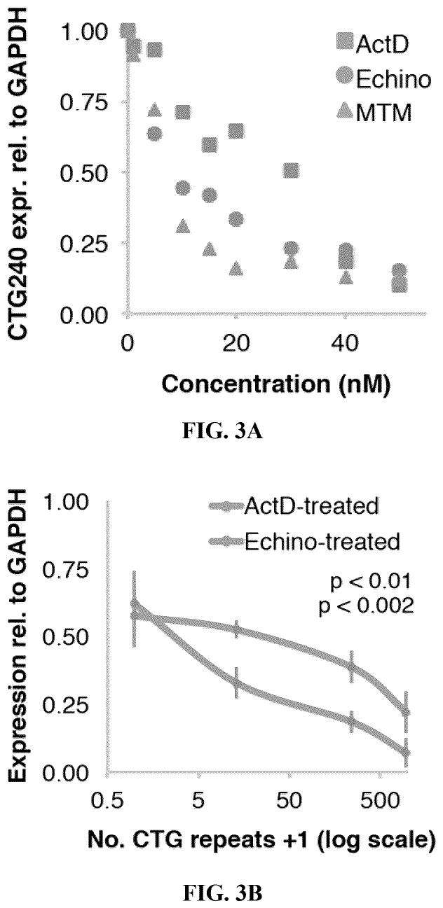

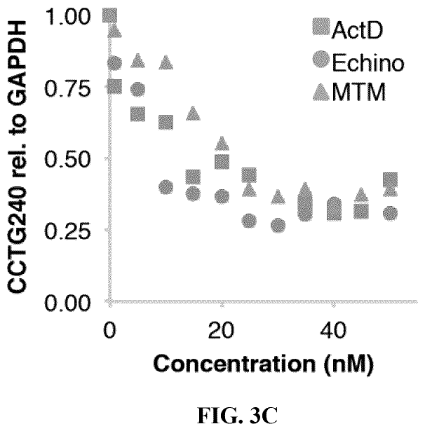

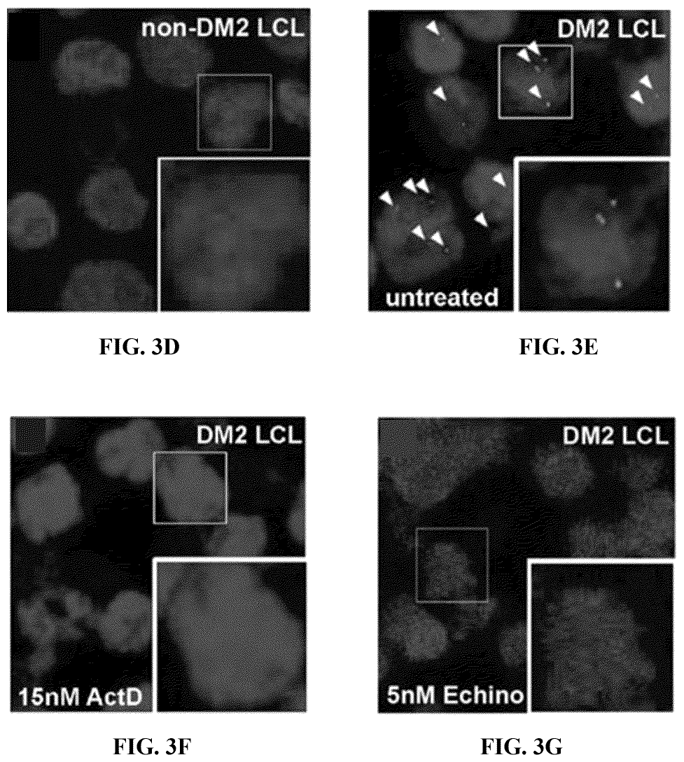

Small molecules were identified that impede transcription of HREs. Although the dCas9/gRNA system could be developed into a gene therapy, there are delivery challenges associated with such a treatment. Therefore, small molecules will be identified that can impede transcription through HRE-containing transcripts. In the DM field, FDA-approved compounds have been identified that can specifically reduce the abundance of CTG and/or CCTG repeat-containing transcripts. For example, at low nanomolar doses, the global transcriptional inhibitor Actinomycin D (ActD) can preferentially impede transcription of expanded CTG repeats; this leads to a reduction in CUG repeat-containing RNA, release of the Muscleblind-like RNA binding proteins, and rescue in RNA mis-splicing in a mouse model of DM¹⁸. Additional FDA-approved compounds have been identified that may inhibit transcription of GC-rich sequences, and their efficacy in reducing CTG/CCTG repeat transcription was tested (FIG. 3). Several compounds do so in a concentration-and repeat length-dependent manner, as measured by RT-qPCR relative to GADPH, as well as by presence of RNA foci in patient cell lines. It will be determined whether the compounds shown in FIG. 3 also inhibit transcription of G₄C₂ and C₄G₂ repeats, as well as adapt a high throughput screen that was developed for DM1 CTG repeats to HREs. In brief, a HeLa nuclear extract is used to transcribe repeat-containing transcripts and relevant control transcripts in vitro, and conditions are adjusted to measure abundance of newly transcribed RNA using multiplex RT-qPCR. This system will be adapted so that HREs are transcribed instead of CTG repeats, and FDA-approved compound libraries (e.g., LOPAC) will be screened, as well as additional proprietary libraries. These experiments will synergize with experiments in the context of DM1 and DM2—specifically, compounds may be identified that successfully impede transcription of all expanded repeats, or different compounds whose differential inhibitory activity may be identified that will enlighten the structure-function relationships for multiple repeat sequence types. Compounds identified will be tested for activity and toxicity in C9ALS/FTD fibroblasts and iPS-derived neurons. If these compounds are active in cells and have low toxicity, they will then be tested in the BAC-based mouse model described below.

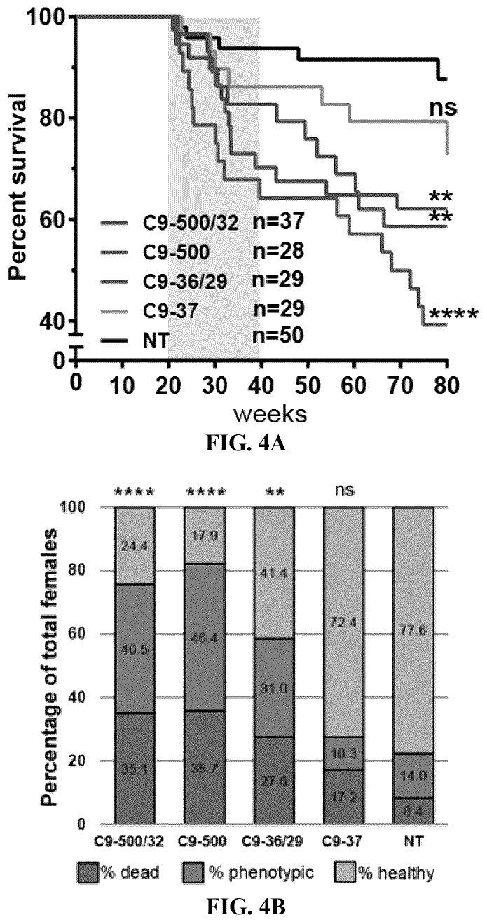

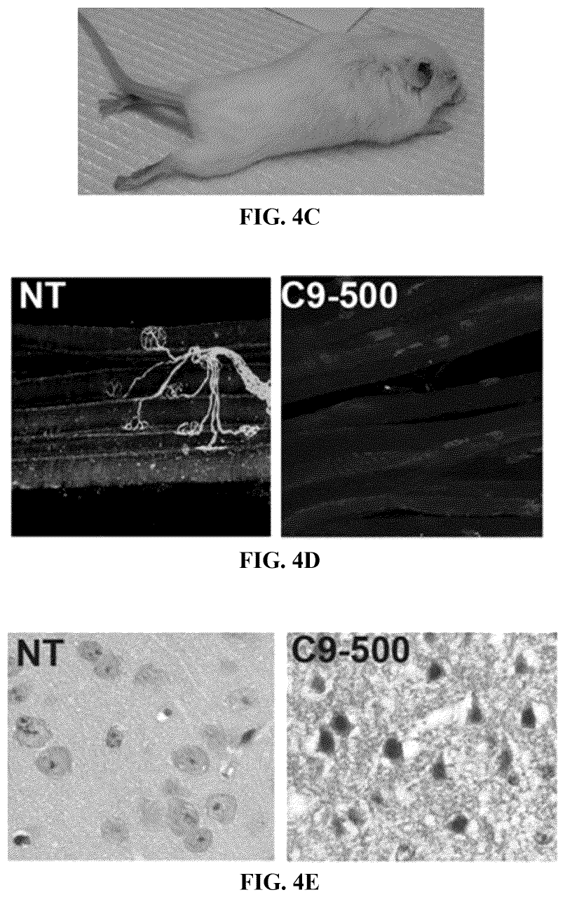

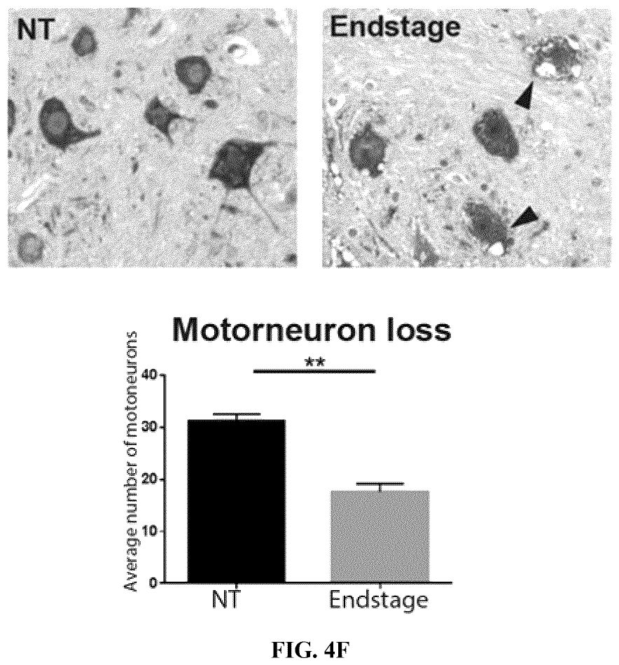

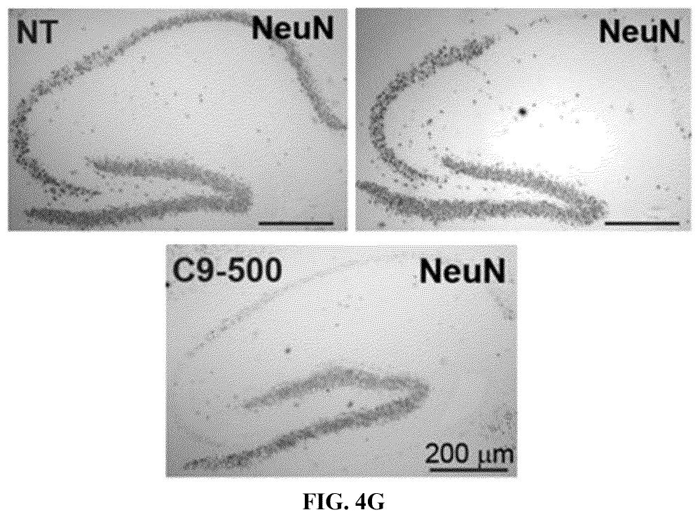

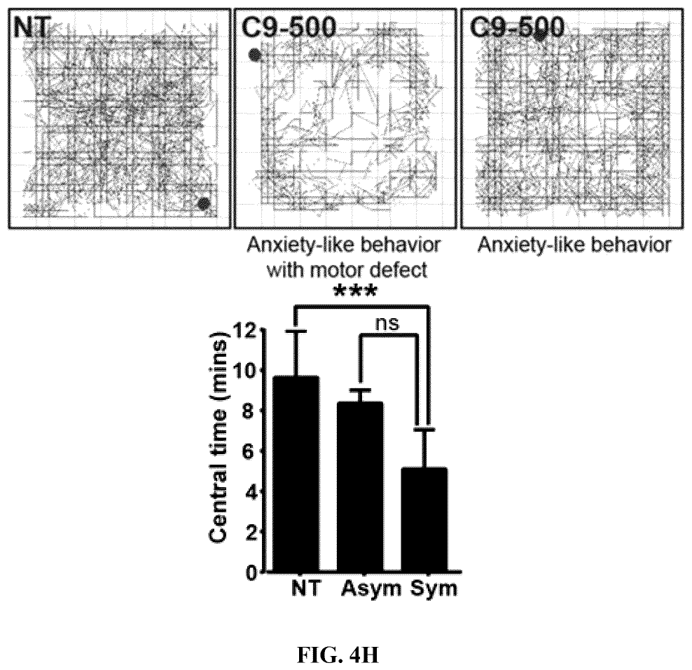

Changes in a mouse model of C9ALS were characterized. A BAC-based model that recapitulates molecular, neurodegenerative and behavioral changes observed in C9 ALS/FTD patients was developed. Three independent lines, generated using a patient-derived C9ORF72(+) BAC, develop molecular and clinical features of ALS/FTD in a repeat-length and gene-dose dependent manner, including a single copy line with 500 G₄C₂ repeats (C9-500). Both sense and antisense RNA foci accumulate in expansion animals and are found throughout the brain and spinal cord. Additionally, the three symptomatic lines express GA and GP RAN proteins, which accumulate as aggregates in the brain. All three symptomatic lines develop neurological/neuromuscular phenotypes starting at ˜4 months including: a) limb paralysis; b) neuromuscular junction denervation; c) neuronal loss in multiple brain regions including spinal-cord lower motor neurons, layers II/III and V of the motor cortex; d) anxiety-like behavior and e) decreased survival (FIG. 4). In summary, these mice recapitulate molecular and severe, fatal neurodegenerative features of C9ORF72 ALS/FTD and provide an important tool to test the therapeutic efficacy of CRISPRi on C9 ALS/FTD.

Dosing and safety: Initial tests will be performed to assess dosing and safety of AAV-dCas9/gRNA to transduce the CNS of wildtype (FVB) mice via retro-orbital intravenous injections. The viral capsids, PHP.A and PHP.B, will be used to efficiently transduce the central nervous system, including neurons, glia, and oligodendrocytes of the brain and spinal cord. A small cohort of mice (n=4 mice per time point) will be injected with various doses of AAV dCas9/guide RNA, monitored for adverse reactions, and harvested at 2 or 4 weeks post-injection. Frozen and formalin-fixed paraffin embedded brains and spinal cords from these mice will be examined by immunohistochemistry (IHC), immunofluorescence (IF) and histopathology for expression of dCas9 and/or any associated CNS damage. Off-target effects on gene expression and RNA splicing will be monitored by RNAseq and will serve as a baseline for analysis in the C9-500 mice. The dosage of dCas9/guide RNA that gives the best CNS penetration and persistence with the least off-target effects will be selected for analysis in C9-500 BAC mice.

Timing of treatment: Disease onset in C9-500 mice is detectable at ˜4 months of age, via DigiGait analyses of hindlimb gait abnormalities, prior to overt phenotypes, such as paralysis, weight loss and death. At ˜5 months the mice develop overt disease phenotypes, including a slow progressive or an acute rapid-onset phenotype. By one year, 82% of mice from this line are phenotypic: 46% with overt cage-behavior abnormalities and 36% have died. The severity and lethality of the disease in these mice, coupled with the ability to detect onset prior to a rapid decline in health, provides an opportunity to test whether CRISPRi can prevent disease progression in a cohort of animals with early features of disease. A cohort of C9-500 BAC mice (n=20 per treatment group) will be treated at the beginning of disease onset (˜4 months) with AAV dCas9 and HRE-targeting gRNA, or control gRNA. A small cohort (5 mice) will be sacrificed at 6 months, and others aged until 10 months. Brain, spinal cord and other tissues will be harvested from animals and either stored fixed or frozen for analyses. During the course of the experiment, animals will be monitored weekly for signs of disease progression (weight loss, limb paralysis, cage behavior) and tested at 4, 6, 8, and 10 months by DigiGait and open field analyses. C9-500 mice spend significantly shorter time in central region of the test chamber. Based on experience with the C9-500 mice, it is expected that roughly 40% of the untreated animals will develop an acute rapidly progressing phenotype over the course of the experiment and that will enable detection of a change in survival of 10% using 20 animals per treatment group. Tissues will also be harvested from mice that reach end-point due to disease during the length of the experiment. All mice will be assigned to treatment groups in a random manner, and scored and treated in double-blinded fashion.

Measuring molecular changes: RAN proteins expressed in these mice accumulate as aggregates in the brain. These aggregates are more abundant in end-stage and older symptomatic mice than in presymptomatic mice. RAN protein accumulation will be quantitated by IHC, IF and ELISA in the brain and spinal cord to determine whether CRISPRi alters the spatial and temporal distribution of RAN protein accumulation. Analysis of RAN protein accumulation following both short (˜2 months of treatment/6 month mice) and long (˜6 months of treatment/10 month mice) treatment will determine whether RAN accumulation is halted and/or reversed by CRISPRi. In parallel, frozen brain and spinal cord sections (5-10 μm) will be examined for sense and antisense foci, as well as RNA levels by qRT-PCR, at each time point. Impacts on the transcriptome will also be measured using RNAseq. Results will be compared to existing RNAseq data from patient tissues.

Measuring neurodegeneration: Neurodegeneration will be monitored at both 6 and 10 months. Neuromuscular junction abnormalities and neuronal loss in multiple brain and spinal cord regions will be monitored by IHC/IF. Because cortical layers II/III and V undergo dramatic neurodegeneration in untreated animals, this area will be used to assess whether CRISPRi reduces neurodegeneration. Differences in the distribution and appearance of RAN-positive cells relative to signs of neurodegeneration will be assayed by IHC/IF double-labeling methods and stereological techniques. Penetration of the dCas9/guide RNA will also be confirmed via IF, IHC and ELISA. Disease progression will be monitored by weight loss, DigiGait, and open field analyses. The efficacy of CRISPRi to prevent acute disease progression and increase survival will be evaluated. For all experiments, the mice will be evaluated in a blinded manner as to genotype and treatment group. Small molecules identified that impede HRE transcription can be analyzed in similar in vivo studies.

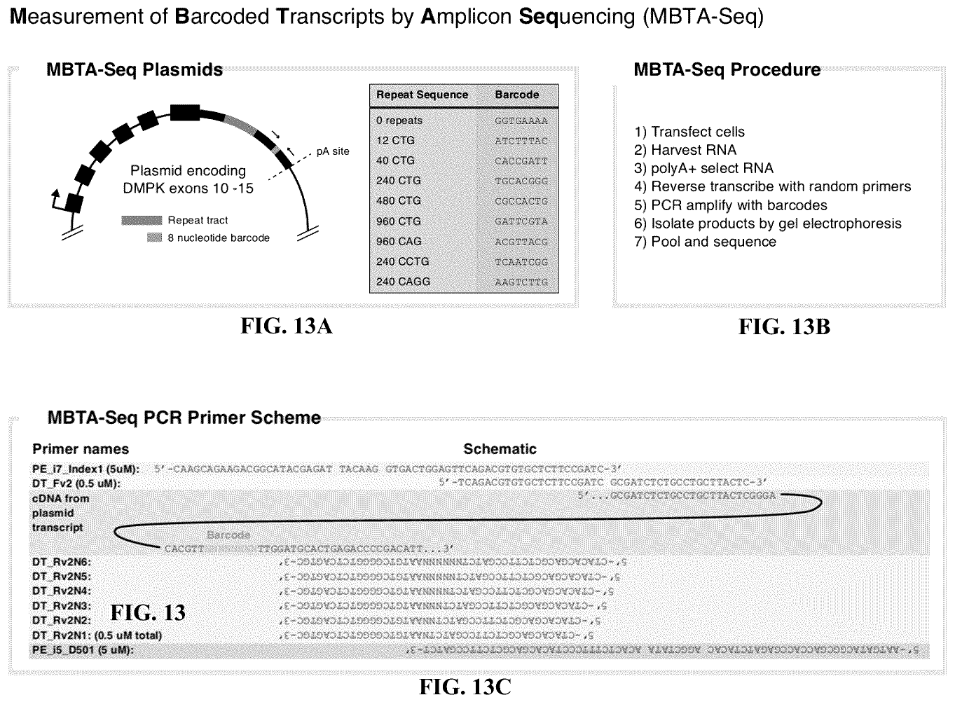

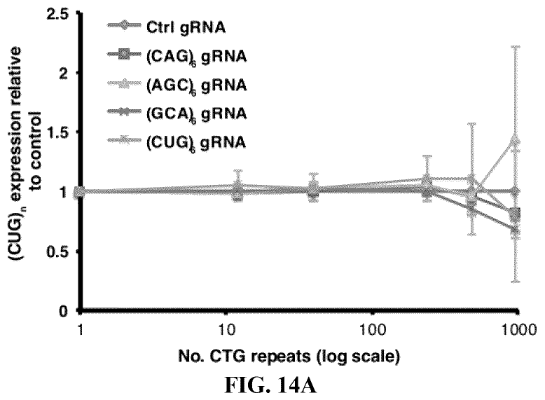

dCas9-gRNA complexes reduce abundance of repeat-containing RNAs in a length-, PAM-, and strand-dependent manner. dCas9 impedes transcription of expanded microsatellite repeats more potently than non-repetitive sequences because expanded repeats may: 1) present challenges for RNA Pol II elongation even under normal conditions; and 2) allow for high levels of dCas9 recruitment using a single guide RNA (gRNA) sequence, forming a substantial block to the elongating polymerase (FIG. 7A). A plasmid-based strategy was employed in cell culture to examine the effects of dCas9 recruitment on transcription of CTG/CAG repeats that occur in DM1, FECD, HD, and SCAs. Plasmids containing 0, 12, 40, 240, 480, or 960 CTG repeats located >1.5 kilobases downstream of the TSS in the DMPK 3′ UTR were utilized. To recruit dCas9 to these repeats, gRNAs targeting CTG/CAG repeats in each of 3 possible nucleotide phases of the non-template strand, and 1 phase of the template strand were designed (FIG. 7B, top). Each PAM was, therefore, constrained to CAG, AGC, GCA, or CTG. An amplicon-based deep sequencing assay called “Measurement of Barcoded Transcripts by Amplicon Sequencing” (MBTA-Seq) was developed to precisely measure the expression of the repeat transcripts (FIG. 13). For this assay, plasmids were modified by introducing distinct 8 nucleotide barcodes downstream of each CTG repeat tract. RNA was harvested from cells transfected with these plasmids, polyA+ selected, amplified by RT-PCR across the barcode region but avoiding the repeat containing region, and deep sequenced (FIG. 13). This assay was highly reproducible and allowed quantitation of multiple repeat-containing transcripts in the same pool of cells. In the presence of dCas9 and (CAG)₆ gRNA, a dramatic reduction in expression of RNAs containing expanded CUG repeats was observed (FIG. 7B, bottom). Knockdown efficiency increased with the number of repeats, presumably due to the increased number of dCas9-gRNA complexes recruited. Twelve CTG repeats, likely recruiting at most a single dCas9-gRNA complex, showed −50% repeat-containing RNA remaining, and 40 CTG repeats showed −25% remaining. Repeat lengths >240 CTG showed only −5% remaining. (AGC)₆ and (GCA)₆ gRNAs showed poor knockdown efficiency, consistent with previously described SpCas9 PAM preferences, where NGG is best, NAG is second best, and NCG/NTG are equally disfavored. (CUG)₆ gRNA showed little to no knockdown of transcripts containing expanded CUG repeats, potentially due to a weak PAM as well as targeting to the template strand. Importantly, dCas9 was required for decreased expression of repeat-containing transcripts, as presence of gRNAs alone did not lead to knockdown (FIG. 14A).

These observations suggest that recruitment of dCas9-gRNA complexes can impede transcription of expanded microsatellite repeat tracts in a repeat length-dependent manner. However, these experiments were performed using transiently transfected plasmids, which may not accurately model all aspects of transcriptional regulation in a genomic context. Furthermore, experiments to assess binding of protein complexes to DNA loci are most commonly performed using genomic targets and not plasmids. Therefore, a HeLa cell line was established in which transgenes for each of the 6 CTG repeat lengths, with associated barcodes, were stably integrated (FIG. 14B). To measure DNA binding by dCas9-gRNA complexes, chromatin immunoprecipitation (ChIP) was performed against dCas9 in the presence of (CAG)₆ gRNA or non-targeting control gRNA. Similar to MBTA-Seq, the barcodes were deep sequenced to quantitate abundance of immunoprecipitated DNA encoding each repeat length. To confirm transcriptional repression in this cell line, RNA abundance was measured by MBTA-Seq following transfection of dCas9-gRNA complexes. It was observed that dCas9-gRNA binding to DNA increased as a function of repeat length, concomitant with a decrease in relative RNA abundance, when comparing (CAG)6 gRNA to control (FIG. 7C). In a genomic context, dCas9-gRNA reduced the abundance of transcripts containing 960 CUG repeats to ˜20%, and was associated with ˜8-fold increased binding relative to genomic loci without CTG repeat tracts. These observations suggest that binding of dCas9-gRNA complexes to DNA impedes transcription of repeat tracts, and that both binding and potency of transcriptional inhibition increases with the number of repeats.

To test whether dCas9-gRNA complexes can impede transcription of other expanded repeats, CCTG repeat tracts, which cause DM2 were assayed, and similar reduction in expression of CCUG repeat-containing RNAs was observed (FIG. 7D). Here, the tetranucleotide repeat allowed testing of 4 distinct gRNAs targeting the non-template strand, (CAGG)₅, (AGGC)₅, (GGCA)₅, and (GCAG)₅, corresponding to AGG, GGC, GCA, and CAG PAMs, respectively. Consistent with PAM preferences of SpCas9, knockdown was most efficient with the (CAGG)₅ gRNA, although the (AGGC)₅ and (GCAG)₅ gRNAs were also quite effective, in contrast to the (CCUG)₅ gRNA. Similar to studies of expanded CUG repeats, presence of gRNAs alone did not lead to knockdown (FIG. 14C).

To study how the PAM and targeted strand separately influence the efficacy of transcriptional blockade, and to clarify DNA versus RNA-targeting mechanisms, (CAG)₉₆₀ and (CAGG)₂₄₀ plasmids suitable for MBTA-Seq were generated. These constructs are identical to their (CTG)₉₆₀ and (CCTG)₂₄₀ repeat-containing counterparts except for the repeat tract and barcode. MBTA-Seq was then used to measure the efficiency of CUG₉₆₀ or CAG₉₆₀ knockdown in the presence of either (CAG)₆ or (CUG)₆ gRNA (FIG. 7E). By testing all four combinations, PAM-dependent effects were separated from strand-dependent effects. Targeting the non-template DNA strand reduced expression more effectively than targeting the template strand. Specifically, (CUG)₉₆₀ was reduced to −5% by a (CAG)₆ gRNA, while (CAG)₉₆₀ was reduced to −40% by the same gRNA. These two conditions effectively compare targeting of non-template vs. template strand, controlling for the CAG PAM. Similarly, (CAG)₉₆₀ was reduced to −50% by the (CUG)₆ gRNA, and (CUG)₉₆₀ remained relatively high at −97%. Again, targeting of the non-template strand was more efficient than targeting of the template strand, controlling for the CTG PAM.

PAM-dependent effects were quantified by comparing abundance of (CTG)₉₆₀ in the presence of (CAG)₆ gRNA relative to abundance of (CAG)₉₆₀ in the presence of (CUG)₆ gRNA. Here, both gRNAs target the non-template strand, but use different PAMs. ˜5% RNA remained with the CAG PAM, and ˜55% RNA remained with the CTG PAM, controlling for targeted strand, consistent with the CAG PAM being more effective. Similarly, measurement of (CAG)₉₆₀ RNA in the presence of (CAG)₆ gRNA and (CTG)₉₆₀ RNA in the presence of (CUG)₆ gRNA allows comparison of both PAMs, controlling for targeted strand. Again, more effective silencing was observed with a CAG PAM (˜40% RNA remaining) as compared to the CTG PAM (no change in RNA). Similar trends were observed with CCTG/CAGG repeats (FIG. 7F). (CCUG)₂₄₀ was reduced to ˜5% by (CAGG)₅ gRNA and (CAGG)₂₄₀ to ˜55% by (CAGG)₅ gRNA (non-template vs. template strand, AGG PAM). Although the same template vs. non-template effect was not observed when silencing with a CTG PAM, the (CCTG)₅ gRNA was largely ineffective, showing little to no silencing.

Overall, these results separate the effects of PAM sequence and targeted strand in the context of transcriptional blockade. Furthermore, they support a model in which dGas9/gRNA complexes target repeat-containing DNA, because reductions in RNA abundance are achieved even when using gRNAs that are not complementary to transcribed RNAs.

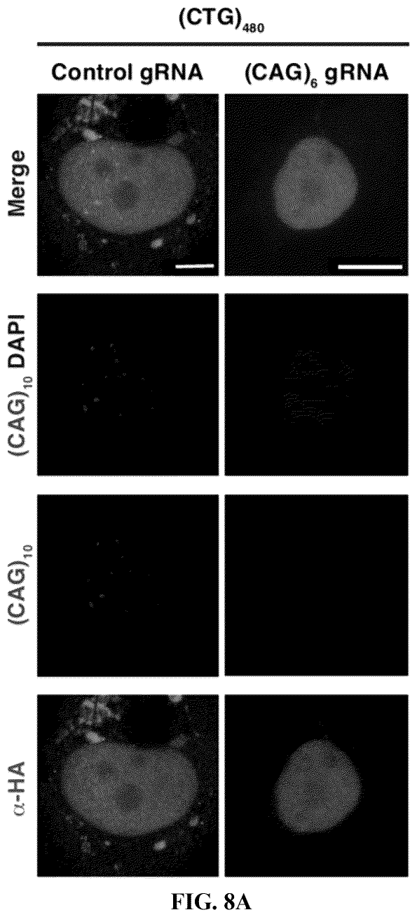

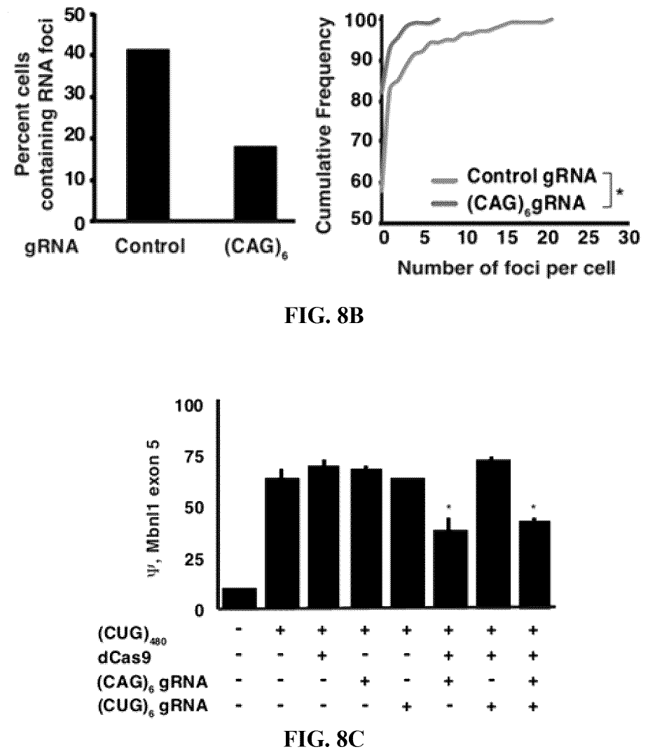

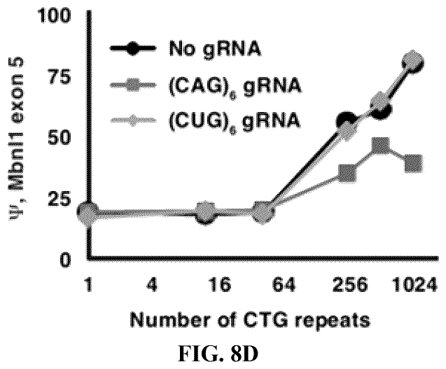

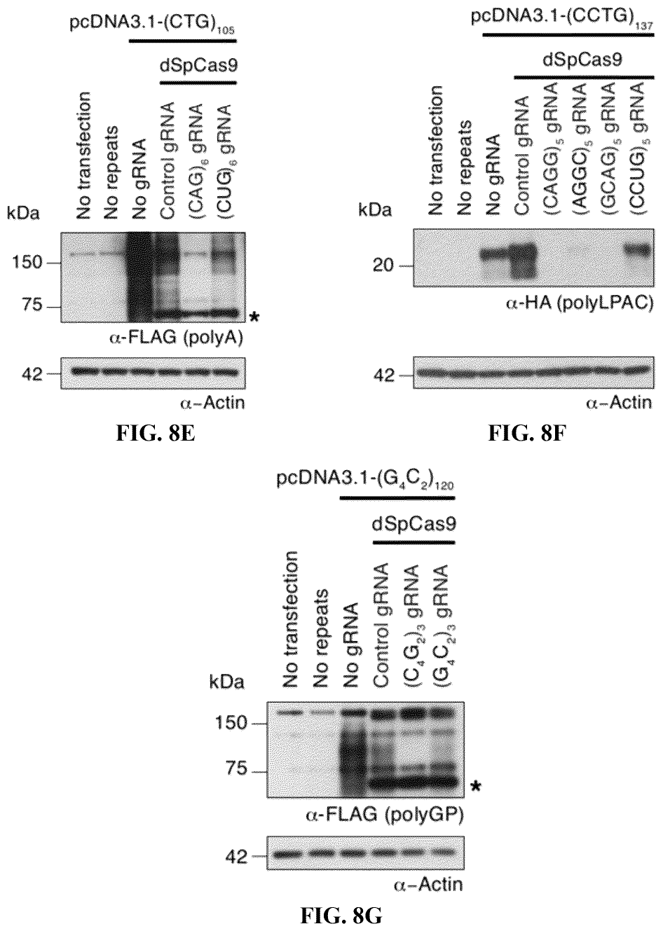

dCas9-mediated transcriptional inhibition rescues splicing defects and blocks RAN translation in cell-based models of DM and C9ALS/FTD. Downstream symptoms of many repeat expansion diseases are caused by the expression of toxic RNA species, which sequesters RNA binding proteins necessary for cellular functions, causing downstream changes to the transcriptome and proteome. To assess whether dGas9-mediated transcriptional silencing can rescue molecular and cellular phenotypes in disease models, a HeLa cell-based model of DM1 was used, in which (CUG)₄₈₀ repeats are expressed from a plasmid. Co-expression of dCas9 and (CAG)₆ gRNA led to a reduction in the percentage of cells showing CUG-containing RNA foci, as well as a reduction in the number of foci per cell (FIG. 8A, B). Rescue of MBNL-dependent splicing misregulation, characteristic of DM1 cells was measured, using a splicing minigene reporter containing MBNL1 exon 5. The percent spliced in (psi, Ψ) of this exon is regulated by MBNL proteins, and changes from 10% in healthy HeLa cells to −70% in cells expressing (CUG)₄₈₀repeats. Co-transfection of dCas9 and (CAG)₆ gRNA partially rescued splicing dysregulation, reducing Ψ to −35% (FIG. 8C). This rescue required both dCas9 and (CAG)₆ gRNA, indicating that (CAG)₆ gRNA expression alone cannot neutralize toxic CUG RNA by displacing MBNL protein or facilitating degradation. Splicing defects in DM1 depend on total CUG repeat load; Ψ values for MBNL1 exon 5 increased with the length of the CTG repeat tract transfected into cells (FIG. 8D). Interestingly, co-expression of dCas9 and (CAG)₆ gRNA yielded a similar rescue of splicing across all repeat lengths exhibiting mis-splicing, i.e. 240, 480, and 960 CUG repeats.

The CTG, CCTG, and G₄C₂ repeats associated with DM1, DM2, and C9ALS/FTD, respectively, undergo repeat-associated non-ATG (RAN) translation. dCas9-mediated transcriptional repression could also yield reduced RAN protein abundance. Western blot analyses of RAN peptides, facilitated by downstream protein tags, revealed reduction in CTG, CCTG, and G₄C₂ RAN product (FIG. 8E). In all three western analyses, RAN proteins were observed only in with repeat-containing plasmids, and were slightly reduced upon expression of a control, non-targeting gRNA, potentially due to competition of plasmids for transcriptional machinery. Consistent with measurements of CUG-containing RNA levels (FIG. 7B), the FLAG-tagged poly-Ala peptide translated from CUG repeats was dramatically reduced in the presence of (CAG)₆ gRNA, and modestly reduced in the presence of (CUG)₆ gRNA (FIG. 8E). Importantly, RAN protein was not reduced in the presence of (CAG)₆ gRNA alone without dCas9 (FIG. 14D). HA-tagged poly-LPAC translated from CCUG repeats was dramatically reduced in the presence of (CAGG)₅, (AGGC)₅, and (GCAG)₅ gRNAs (FIG. 8F), but not in the presence of (CCUG)₅ gRNA (FIG. 14E). To test effects of dCas9 on levels of poly-GlyPro translated from G₄C₂ repeats, two gRNAs were designed with NGG PAMs: (C₄G₂)₃ that would target the non-template strand and (G₄C₂)₃ that would target the template strand. Consistent with studies of CTG and CCTG repeats, dCas9-gRNA complexes targeted to (G₄C₂)₁₂₀ exhibited silencing of RAN peptide in a strand-dependent manner (FIG. 8G), and did not occur in the presence of gRNA alone (FIG. 14F). While reduction in RAN protein was achieved with both gRNAs, silencing was more effective with gRNA targeting the non-template strand.

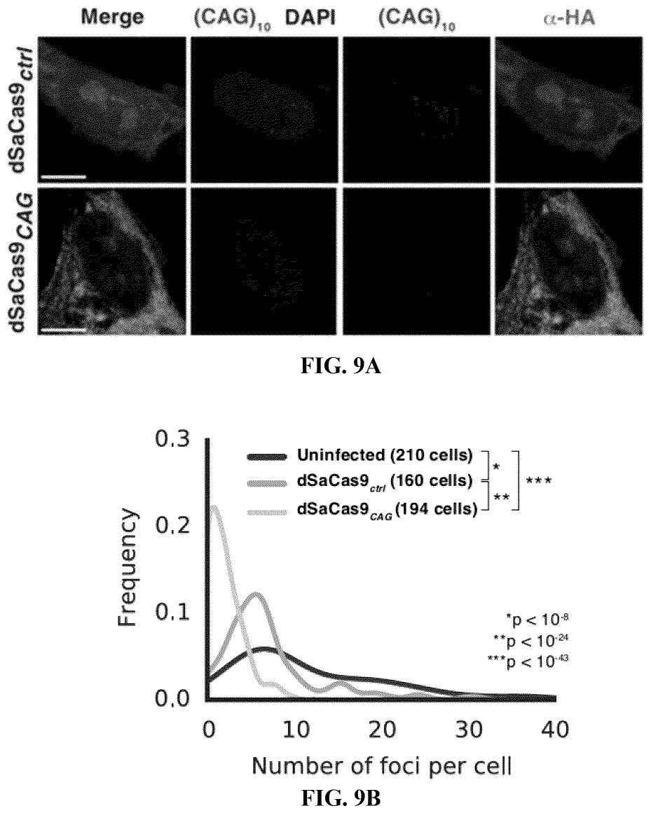

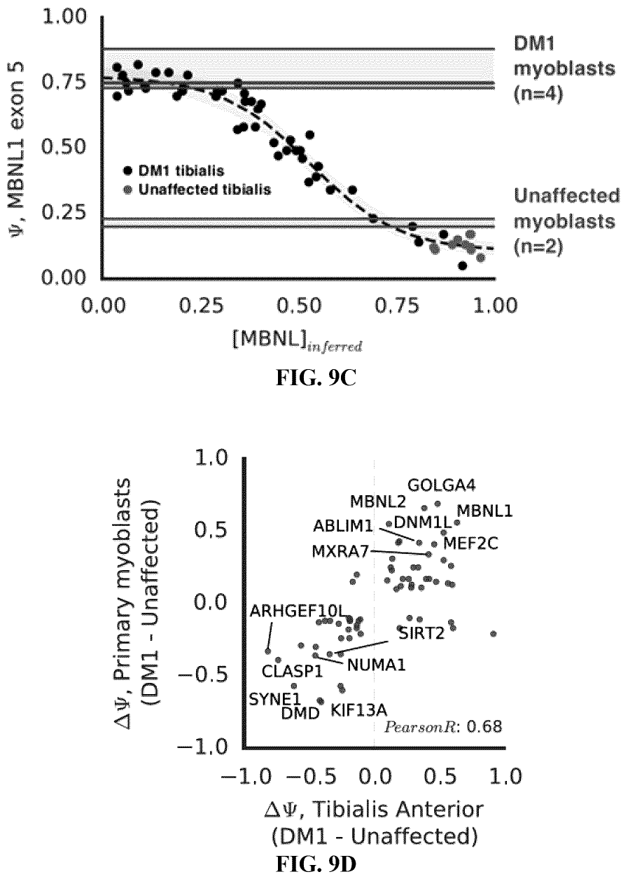

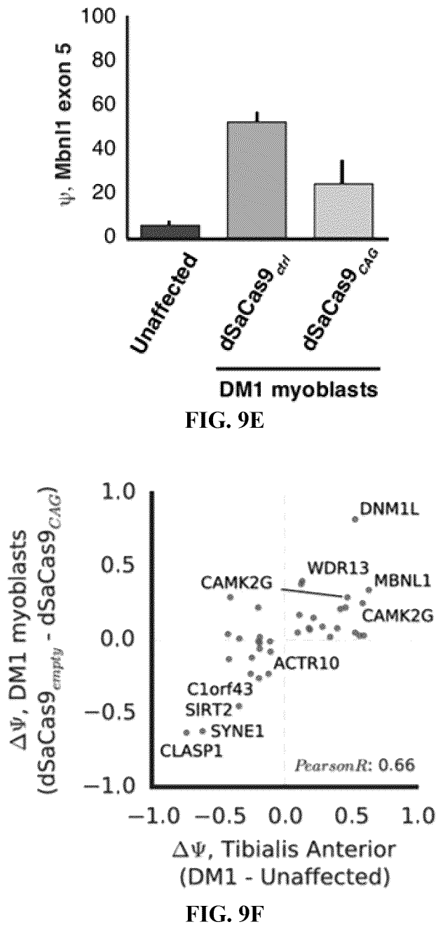

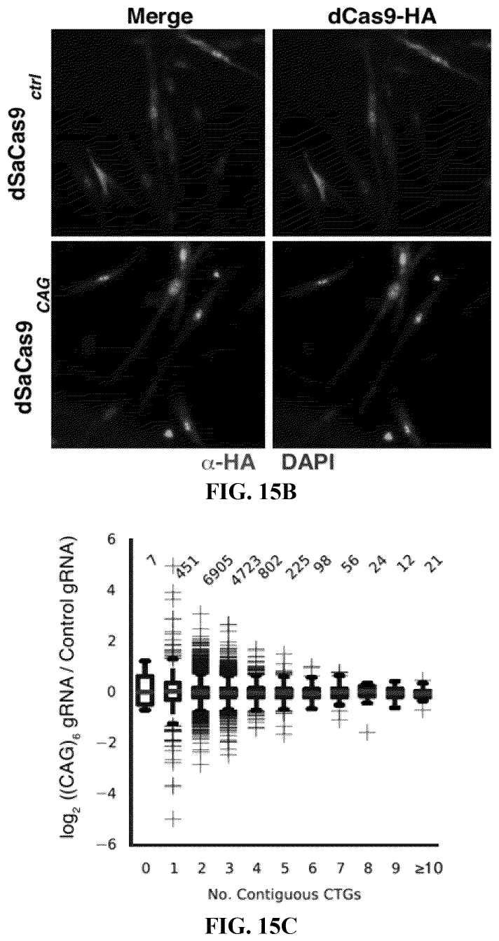

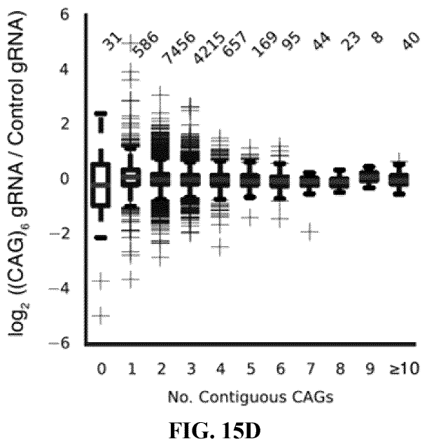

dCas9-mediated transcriptional inhibition reduces nuclear RNA foci and rescues splicing defects in human DM1 myoblasts. Whether this approach could impede transcription of expanded repeats in the native DMPK locus was tested in primary human DM1 myoblasts by delivering dCas9 via AAV. Due to AAV genome size limits, the smaller S. aureus Cas9 (SaCas9) was packaged with a U6 promoter-driven gRNA and deactivated via D10A and H557A mutations. Since dSaCas9 exhibits PAM preferences distinct from dSpCas9 (NNGRR versus NGG), MBTA-Seq was used to confirm that dSaCas9 with a (CAG)₆ gRNA could impede transcription of expanded CTG repeats in a length-dependent manner (FIG. 15A). Next, dSaCas9 was packaged with control or (CAG)₆ gRNA into AAV2/6, because the AAV6 capsid efficiently infects myoblasts in culture (FIG. 15B). dSaCas9 protein carrying 5 nuclear localization signals was found to be nuclear, although at times also cytoplasmic (FIG. 15B, FIG. 9A). Similar to studies in HeLa, the number of CUG repeat foci per cell was decreased in the presence of dSaCas9 and (CAG)₆gRNA relative to control gRNA (FIG. 9A, B).

Although splicing events in DM1 patient muscle have been well characterized, cultured myoblasts do not express many transcripts present in mature muscle. The DM1 myoblasts described above as well as an unaffected line were further characterized to identify molecular changes appropriately modeling in vivo DM1 biology. Four RNAseq libraries were created from the DM1 cells and 2 from the unaffected line, each grown in low or high serum conditions. To identify splicing changes appropriately modeling those observed in human DM1 muscle, a high confidence set of DM1-relevant splicing events was defined by re-analyzing a set of 55 transcriptomes (44 DM1 and 11 unaffected) from human tibialis biopsies (GSE86356). Splicing events were identified whose inclusion level, Ψ, strongly correlated to the concentration of free, functional MBNL protein in the affected tissue. Sigmoid curves were fit describing the relationship between MBNL concentration and Ψ, for example for MBNL1 exon 5 (FIG. 9C). The best-fitting events were selected (see Examples), and their dysregulation (ΔΨ, Tibialis anterior, DM1 minus unaffected) was plotted against dysregulation observed in the DM1 myoblasts (ΔΨ, Primary myoblast, DM1 minus unaffected) (FIG. 9D). These filtering steps yielded a set of myoblast splicing events that models splicing events in human DM1 muscle.

To assess splicing rescue by dCas9, RT-PCR was performed to measure the inclusion level of MBNL1 exon 5. While viral infection with an AAV encoding dSaCas9 and control gRNA yielded a Ψ of ˜50%, infection with AAV encoding dSaCas9 and (CAG)₆ gRNA rescued Ψ to ˜20% (FIG. 9E). Splicing transcriptome-wide by RNAseq was assessed, and analysis was performed on those exons in the myoblasts whose changes are concordant with changes observed in tibialis biopsies (lower left and upper right quadrants, FIG. 9D), and whose baseline Ψ values are less than 0.33 units apart when comparing tibialis to myoblasts. Several exons were successfully rescued, and a correlation of ˜0.66 was observed between dysregulation in tibialis (ΔΨ, DM1 minus unaffected) versus rescue by dSaCas9 and (CAG)₆ gRNA (AT, control gRNA minus (CAG)₆ gRNA) (FIG. 9F). To assess potential off-target changes in gene expression for other transcripts with genomic CTG or CAG repeat tracts, unaffected myoblasts were treated with AAV-dSaCas9 and (CAG)₆ gRNA or control gRNA and RNA-Seq was performed. The longest contiguous CTG or CAG repeat tracts in all pre-mRNAs in the human genome were enumerated, and none were found to exceed 24 repeats; maximums were 24 CTG in TCF4 and 22 in AR. No differences in gene expression were observed that depended on the length of repeat tracts, for both CTG or CAG repeats (FIG. 15C, D). These observations suggest that dSaCas9 targeted to CTG repeat tracts can impede transcription of expanded CUG repeats, relieve MBNL sequestration, and restore splicing homeostasis in human cells containing DM1 repeat expansions, with selectivity that depends on the extreme repeat lengths commonly found in symptomatic tissue.

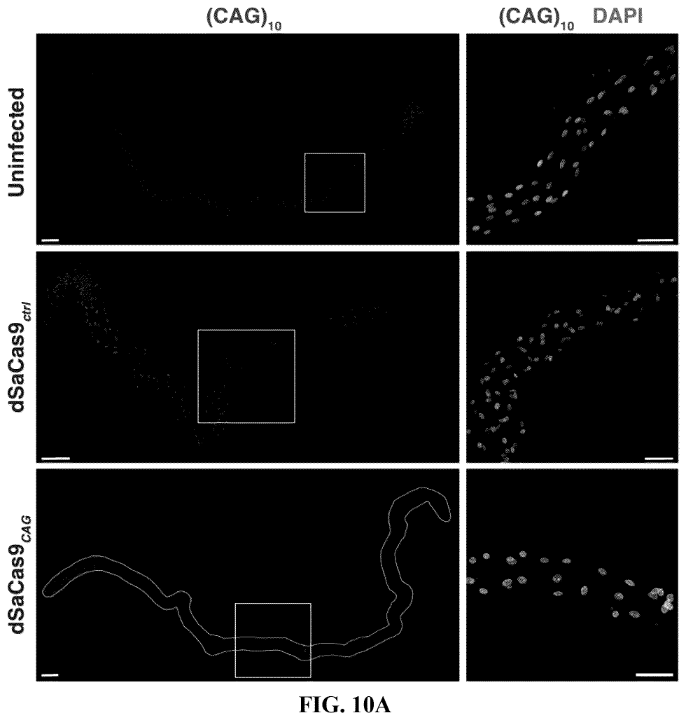

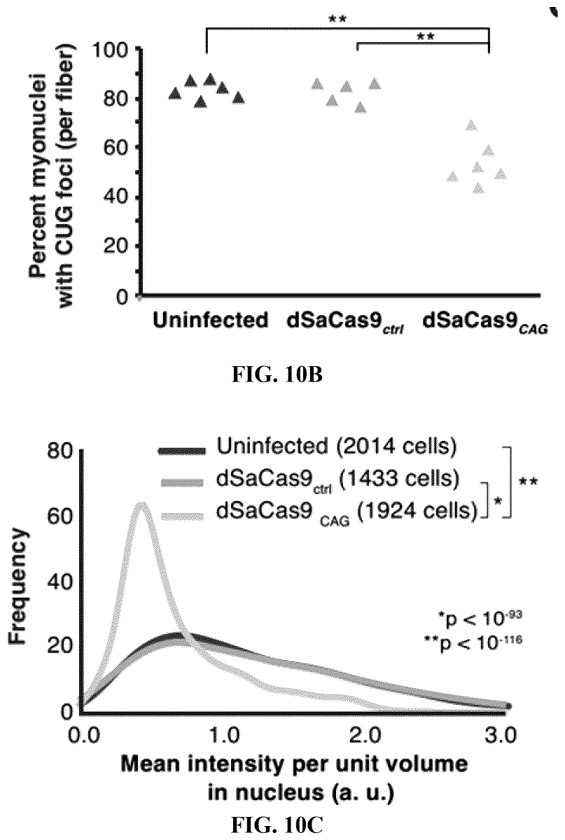

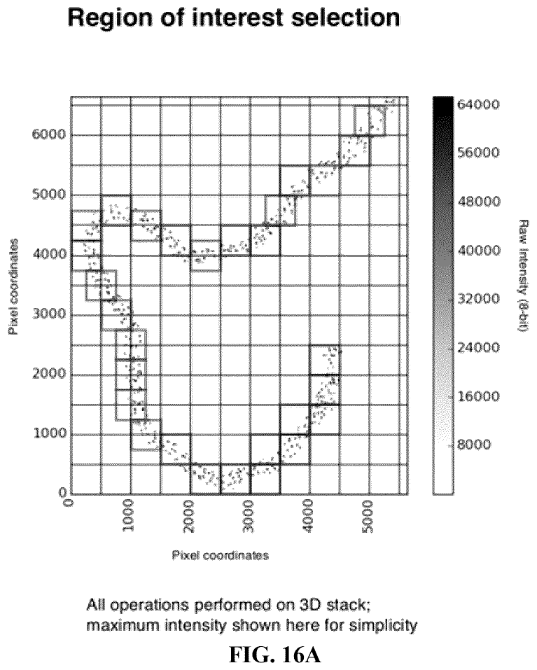

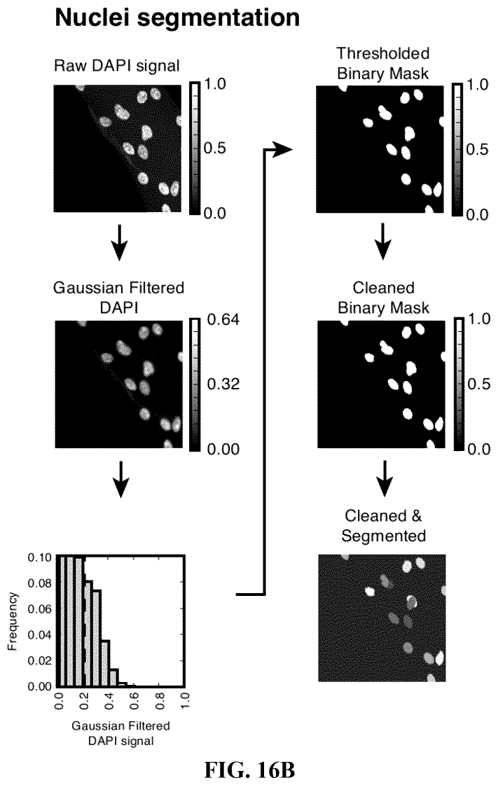

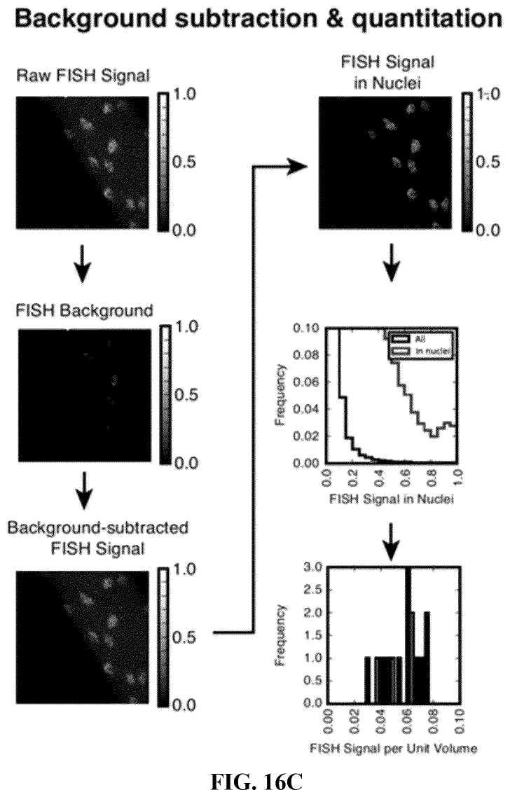

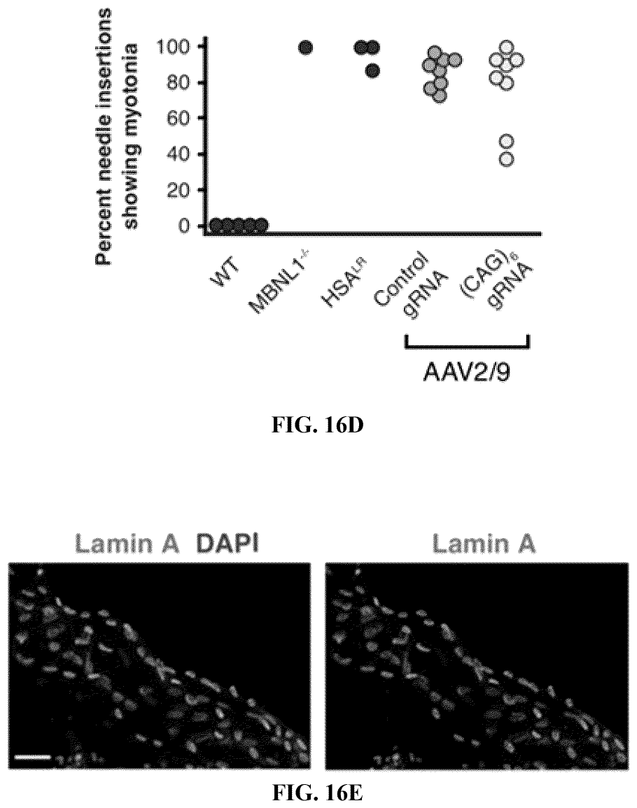

dCas9-mediated transcriptional inhibition reduces RNA foci, rescues Clcn1 expression, and decreases myotonia in a mouse model of DM1. It was assessed whether dCas9 could impede transcription of expanded CTG repeats in a well-established mouse model of DM1, HSA^{LR}. These mice carry a human skeletal actin transgene containing 250 CTG repeats in the 3′ UTR and exhibit molecular, cellular, and phenotypic properties characteristic of DM patients. To assess efficacy of this approach independent of potential in vivo drug delivery challenges, extensor digitorum longus muscle (EDL) fibers were dissected and cultured ex vivo. As CTG repeat expression in this model is driven by the HSA promoter, RNA foci are ubiquitous and numerous in myonuclei, showing FISH signal in ˜80% of myonuclei (FIG. 10A, Top panel). This remained at similar levels following 2 days of AAV treatment with dSaCas9 and control gRNA, but fell to ˜50% with dSaCas9 and (CAG)₆ gRNA. (FIG. 10B). FISH signal intensity, quantitated in >1400 nuclei per condition (see Examples, FIG. 16), was dramatically decreased in fibers treated with dSaCas9-(CAG)₆ gRNA relative to control gRNA and untreated fibers (FIG. 10C). These observations suggest that effective delivery of dSaCas9-(CAG)₆ to muscle fibers ex vivo is sufficient to significantly reduce RNA foci load within 2 days.

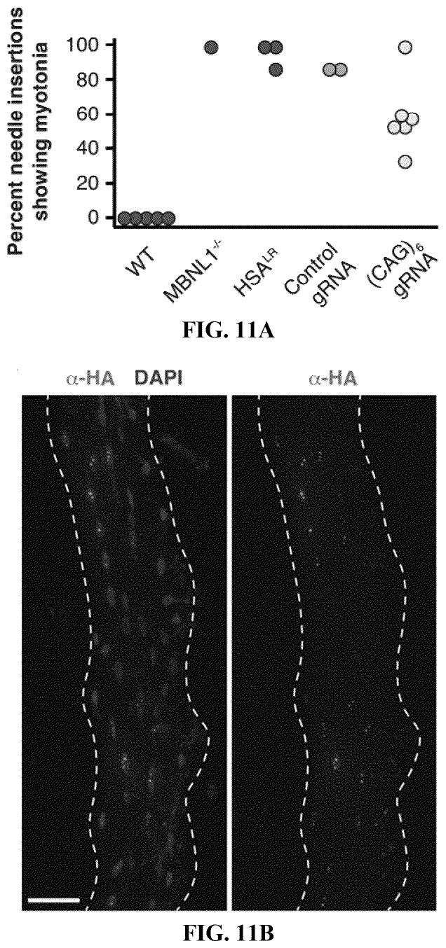

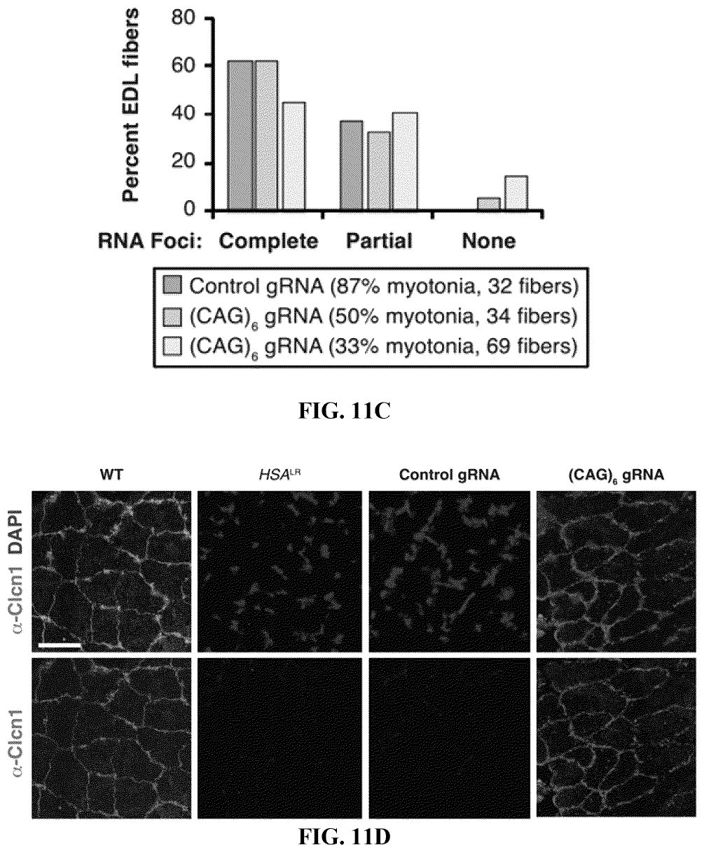

Given previous reports of immune reactivity against Cas9, especially following intramuscular injection, AAV-dSaCas9-gRNA was administered by temporal vein injection at postnatal day 2, prior to full establishment of immune tolerance. AAV2/6 or AAV2/9 carrying dSaCas9 and (CAG)₆ or control gRNA was injected. Five weeks following injection, electromyography was performed to analyze myotonia in tibialis anterior and gastrocnemius muscles (FIG. 11A). Wild type FVB mice showed no myotonia, an Mbnl1 KO mouse showed myotonia in 100% of needle insertions, and HSA^{LR} mice showed myotonia in 87-100% of insertions. HSA^{LR} injected with AAV6-dSaCas9-control gRNA showed myotonia levels similar to uninjected HSA^{LR} (87%). However, HSA^{LR} mice injected with AAV6-dSaCas9-(CAG)₆ gRNA showed a reduction in myotonia, with some showing myotonia in only 33-50% of insertions. Interestingly, nearly all mice injected with AAV2/6 showed rescue while only 1 mouse injected with AAV9 showed rescue (FIG. 16D).

Splicing patterns of Clcn1 mRNA in treated mice were measured because mis-splicing of chloride channel 1 (Clcn1) is well-established to mediate myotonia in DM1 and in HSA^{LR} mice. Surprisingly, when analyzing RNA extracted from bulk muscle tissue, significant rescue in Clcn1 splicing, or other MBNL-dependent splicing events could not be detected. Because myotonia is measured by assaying individual bundles of fibers, it is possible that some regions of muscle may be rescued and not others, and that any potential change in isoform composition in these regions may be diluted in analyses of bulk tissue. This is consistent with a roughly ˜2.5-fold increase in myonuclei per fiber over the first 4 weeks of post-natal development, which could also dilute the proportion of myonuclei containing AAV episomes. AAV6-delivered dSaCas9 distribution in EDL fibers revealed mosaic expression, with rare fibers showing region-specific nuclear signal for dCas9 (FIG. 11B), and most fibers showing absence of dCas9. Control immunofluorescence experiments against lamin A showed ubiquitous and consistent labeling of all myonuclei, ruling out potential staining artifacts (FIG. 16E). In spite of mosaic dSaCas9 distribution, 5-15% of fibers showed complete loss of CUG RNA foci (FIG. 11C). Furthermore, extent of reduction in RNA foci correlated to strength of myotonia rescue across animals. Reduction of RNA foci in some nuclei raised the possibility that proper Clcn1 splicing could be achieved in a subset of nuclei, and that these mRNAs may spread locally throughout the fiber to produce functional Clcn1 protein. Indeed, mice treated with (CAG)₆ gRNA showed increased Clcn1 staining at muscle membranes relative to mice treated with control gRNA (FIG. 11D), but Clcn1 protein was localized only to a subset of fibers per muscle section. These observations are consistent with observations of myotonia elimination in a subset of fibers, yet absence of splicing rescue when assessing bulk tissue. In summary, dCas9 can, in principle, rescue disease phenotypes via transcriptional repression, but widespread rescue of molecular events in muscle will require efficient delivery to a large proportion of myonuclei expressing toxic RNA.

The teachings disclosed herein 1) form the basis for a therapeutic to treat diseases characterized by expanded microsatellite repeats, including ALS, as well as 2) provide important information necessary to benchmark and interpret molecular and physiological effects of this and other related therapeutic strategies. Small molecules that are capable of blocking expanded microsatellite repeat transcription, including HRE transcription, and a gene therapy approach to blocking expanded microsatellite repeat transcription, including HRE transcription, and accurately measuring the effects of blocking expanded microsatellite repeat transcription, including blocking HRE transcription in the context of C9ORF72/ALS/FTD are contemplated.

### Example 1

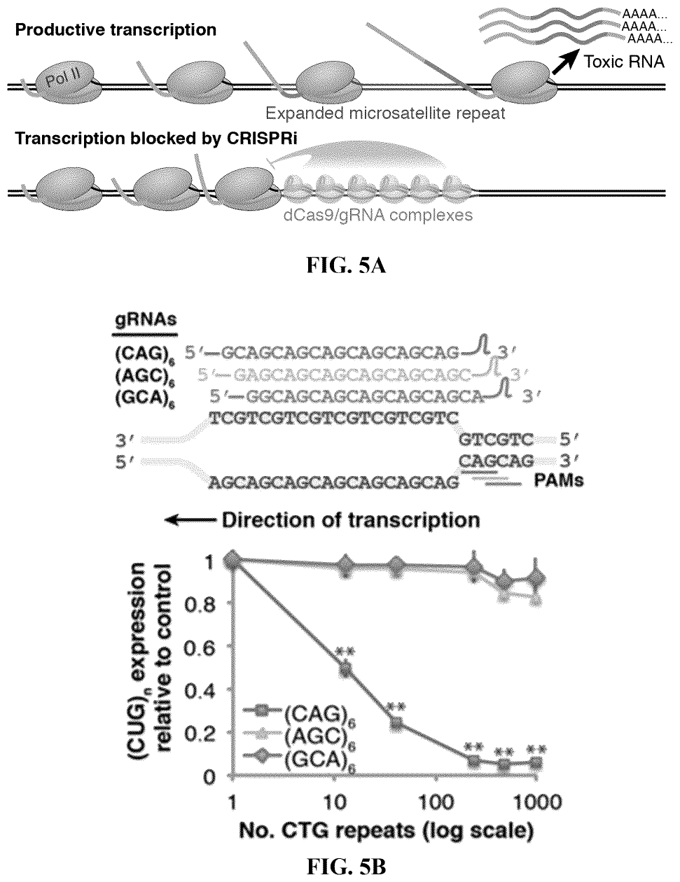

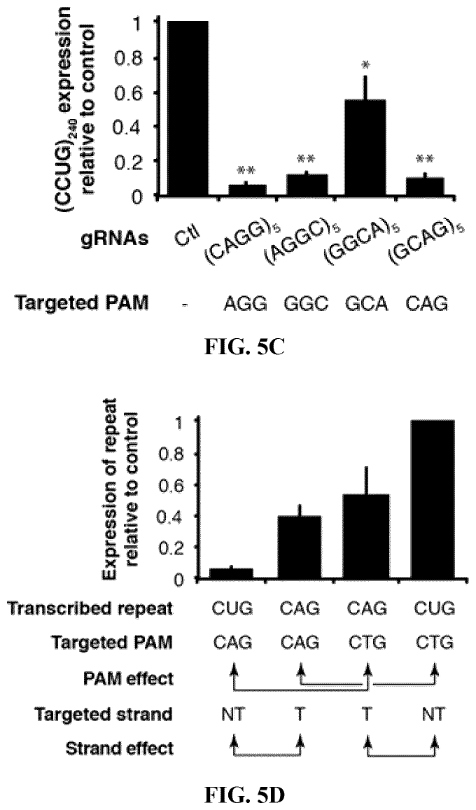

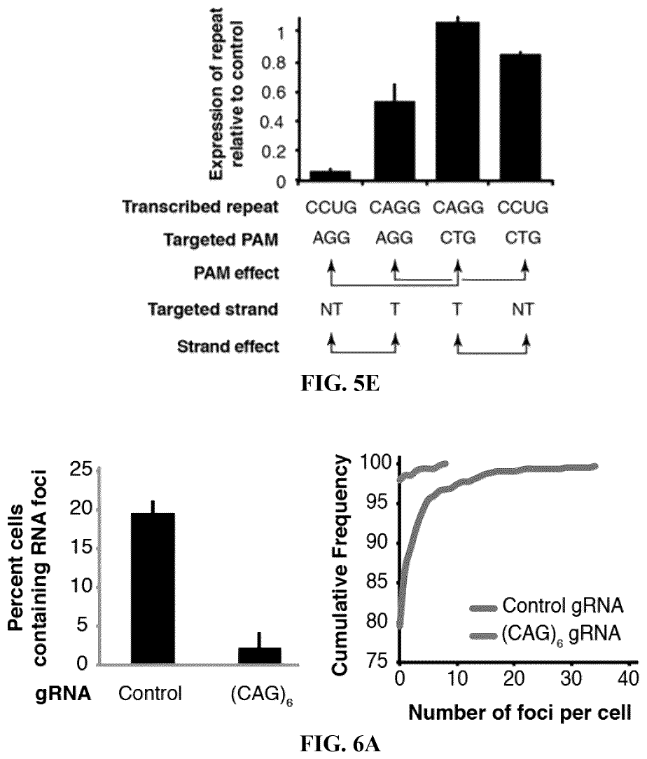

Catalytically deactivated Cas9 (dCas9) inhibits transcription of CTG and CCTG repeat-containing transcripts (CRISPRi). Deactivated Cas9 can inhibit transcription, but typically relies on steric blocking in bacteria or requires a KRAB domain to inhibit initiation in mammalian cells when targeted to promoters. Inhibition of elongating RNA Pol II in mammalian gene bodies showed ˜40% reduction at best. However, long repeats could recruit multiple dCas9 proteins using a single guide RNA (gRNA) sequence, resulting in enhanced steric blockade, consistent with the “CRISPRi collision model”, leading to paused RNAP and disassembly (FIG. 5A). To test this hypothesis, expression of mRNAs containing multiple CUG repeat lengths in the presence of control, CAG-targeting, or CTG-targeting gRNAs was measured. Three CTG-targeting gRNAs were tested, where the register of repeats relative to the PAM (CAG, AGC, and GCA) was shifted (FIG. 5B). A CAG-targeting gRNA was also tested. Consistent with S. pyogenes PAM preferences, the gRNA targeting CAG repeats matching a CAG PAM was highly effective, leading to a repeat length-dependent reduction in expression relative to an mRNA with 0 repeats (FIG. 5B). Similar reduction was observed with expression of 240 CCTG repeats when using guide RNAs targeting (CAGG)₅, (AGGC)₅, (GCAG)₅, but not (GGCA)₅, again consistent with PAM preferences (FIG. 5C). To measure the dependency of transcriptional blockade efficiency on PAM sequence versus targeted strand, expression of plasmids expressing 960 CAG repeats was compared to those expressing 960 CTG repeats, in the presence of (CAG)₆ gRNA or (CTG)₆ gRNA. It was observed that targeting the non-template strand using a CAG PAM was most efficient (FIG. 5D). Similar trends were observed with CCTG/CAGG repeats (FIG. 5E). Similar transcriptional blockade of expanded microsatellite repeats using a deactivated version of S. aureus Cas9 was also observed.

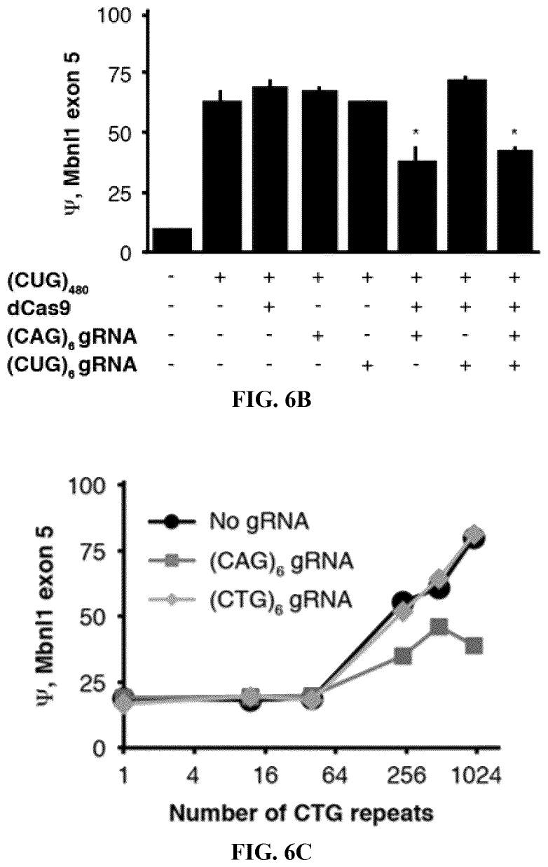

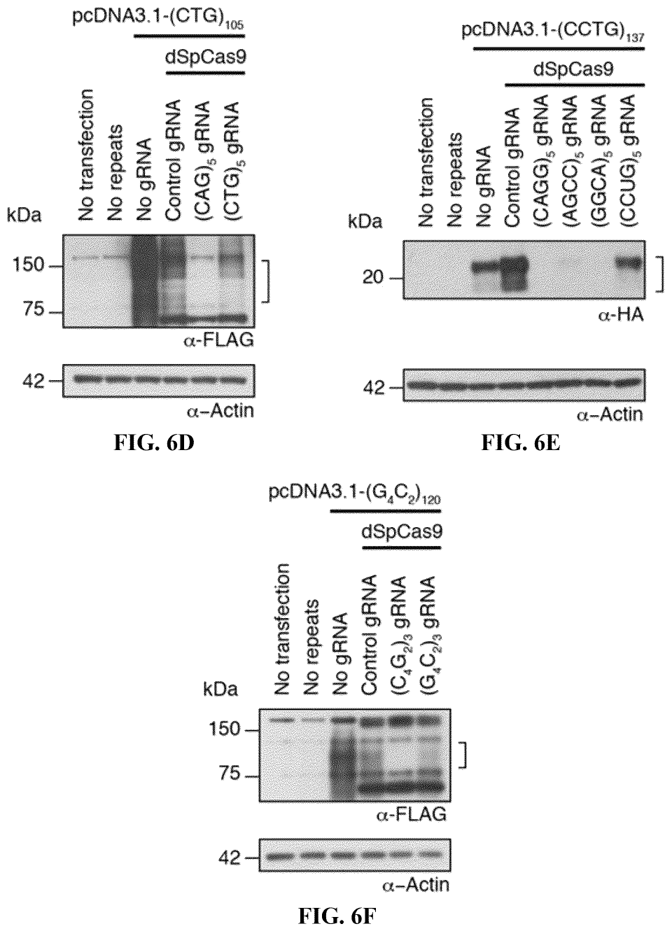

To assess downstream functional consequences of transcriptional blockade in the context of disease models, CUG repeat RNA foci formation was measured in Hela cells transfected with plasmids encoding CTG repeats, in the presence of dCas9 and (CAG)₆ or control gRNA. Targeting dCas9 to repeats led to a reduction in the percentage of cells with RNA foci, as well as the number of foci per cell (FIG. 6A). CUG repeats are known to sequester members of the Muscleblind-like (MBNL) RNA binding proteins, which regulate alternative splicing; by assaying a splicing minigene encoding MBNL1 exon 5, splicing rescue was observed in the presence of dCas9 and (CAG)₆ gRNA (FIG. 6B). Pathogenic splicing was dependent on repeat length, but rescue could be achieved even in the presence of 960 repeats (FIG. 6C). Finally, non-ATG dependent repeat-associated translation, which occurs in the context of expanded microsatellite repeats, was suppressed in the presence of dCas9 and gRNAs targeting appropriate repeat tracts. CTG RAN product, CCTG RAN product, and GGGGCC RAN product were all suppressed by CRISPRi, as assayed by Western blotting of peptide tags downstream of each repeat tract (FIG. 6D-F).

### Example 2: Experimental Model and Subject Details

### Cell Lines

Human cell lines and primary cells were utilized in this study. Patient myoblast cell lines were derived from muscle biopsies under a University of Florida-approved IRB protocol with informed consent from all patients. Information on the genders of the patient cells is unavailable. All cells were grown under standard conditions of 37° C. and 5% CO₂ in media supplemented with 10% fetal bovine serum.

### Animals

In vivo animal studies were performed on HSA^{LR} transgenic mice in this study. Both males and females were used in EMG analysis, which was performed at 5 weeks of age, as by this time point mice display appreciable myotonia. All these studies were performed in accordance with guidelines and regulations of the Institutional Animal Care and Use Committee (IACUC) at the University of Florida.

### Method Details

### Cell Culture, Cell Lines and Transfection

HeLa and HEK293T cells were cultured in 1×DMEM supplemented with 10% fetal bovine serum and 1% Penicillin/Streptomycin at 37° C. and 5% CO₂. The DM1 primary myoblast cells were obtained by G. Xia. The clinical data of research subjects from who muscle biopsies were obtained, have been described before (Xia et al., 2013). Muscle biopsies were performed using 7G UCH Muscle Biopsy Needle (#8066, Cadence Science). Samples were cut into small pieces and seeded into a 6 cm dish. Myoblasts were expanded at a 1:2 ratio in myoblast growth media (Skeletal Muscle Cell Growth Medium-2, Lonza, #CC 3245) at 37° C. and 5% CO₂. For viral transductions, these primary myoblasts were maintained in the growth medium for 3 days and then switched to Differentiation media (DMEM-F12 supplemented with 2% horse serum) for 3 days.

Plasmid DNA transfections of HeLa and HEK293T cells were performed using Trans-IT LT1 (MirusBio) as per manufacturer's instructions. For RNA and FISH analyses, HeLa cells were harvested or processed 72 hours post transfection.

### Cloning Barcoded Repeats and gRNA Plasmids

Non-barcoded plasmids carrying 0, 12, 40, 240, 480, 960 CTG repeats (CTGₙ) and 960 CAG repeats (CAG₉₆₀) were obtained from Tom Cooper (Baylor College of Medicine). Plasmids with 0 and 12 repeats were modified so that their vector backbones were identical to the others, and carried the ampicillin resistance gene. The (CCTG)₂₄₀ plasmid was created as in Philips et al (Philips et al., 1998). In brief, oligonucleotide fragments 5′-TCGA (CCTG)₂₀C-3′ and 5′-TCGAG (CAG)₂₀-3′, were phosphorylated, annealed, gel isolated, and concatemerized by T4 DNA ligase. Concatemers not in a head to tail orientation were digested by SalI and XhoI. Concatemers were gel-isolated and cloned into the SalI site of CTG0. To create CAGG₂₄₀, CTG₉₆₀ was digested with SalI and HindIII and a new fragment was introduced which included HindIII and AgeI restriction sites (DT_MCS). CCTG₂₄₀ was digested with XmaI and HindIII and ligated with DT_MCS digested with HindIII and AgeI to reverse orientation of the repeats and form CAGG₂₄₀.

For MBTA-Seq, each repeat-containing plasmid was barcoded by introducing a fragment containing a random 8-nt sequence at the PflMI restriction site located downstream of the repeats via In-Fusion cloning (Clontech). Clones were sequenced to confirm that each repeat containing plasmid carried a unique barcode (FIG. 13A). Barcoded CTG₉₆₀ and CAG₉₆₀ plasmids were generated by digesting (CTG)₉₆₀ and (CAG)₉₆₀ with HindIII and AccIII and ligating gBlocks containing unique barcodes with the digested plasmids.

The dCas9 gRNAs were cloned into an AflII-digested U6 expression vector by annealing oligos as previously described (Mali et al., 2013). The dSaCas9 gRNAs were cloned into BsaI-digested vector by annealing oligos as previously described (Ran et al., 2015).

### Generating Barcoded Repeat Expressing HeLas

To generate stable cell lines, the DMPK expression cassette was removed from each of the 6 barcoded plasmids and inserted into pAC156 (obtained from Albert Cheng), a plasmid with Piggybac transposon terminal repeats as well as a puromycin selection cassette. All 6 plasmids were transiently transfected together with the Piggybac mPB transposase into HeLa, and selected by puromycin. Single cells were isolated by flow cytometry, and colonies were cultured in 96 well plates. 48 colonies were expanded and subjected to MBTA-Seq to screen for integration and expression of all 6 plasmids.

### dCas9 Chromatin and RNA IP

dCas9 ChIP and RIP experiments were performed on the HeLa cell line containing the six different repeat lengths (CTG0-960) integrated into genome. 10 cm plates of ˜80% confluent cells were transfected with the pXdCas9 and U6 expression vectors expressing the dCas9 protein and the control or (CAG)₆ gRNAs respectively, using the manufacturers guidelines. For ChIP, 48 hours after transfection cells were crosslinked with 1% formaldehyde for 20 minutes at room temperature. Cross-linking reactions were stopped by addition of glycine to a final concentration of 0.125 M. Cells were then harvested, washed with phosphate buffered saline and pelleted. 1 mL of Lysis Buffer 1 (50 mM Hepes [pH 7.5], 140 mM NaCl, 1 mM EDTA, 10% glycerol, 0.5% Igepal, 0.25% Triton X-100) was added to the cells and rocked at 4° C. for 10 minutes. After spinning, the cells were incubated in Lysis Buffer 2 (10 mM Tris-HCl [pH 8.0], 200 mM NaCl, 1 mM EDTA, 0.5 mM EGTA) for 10 minutes at RT. Nuclei were pelleted and resuspended in 1 mL of Lysis Buffer 3 (10 mM Tris-HCl [pH 8.0], 100 mM NaCl, 1 mM EDTA, 0.5 mM EGTA, 0.1% Na-deoxycholate and 0.5% N-lauroylsarcosine) and subjected to sonication in a Covaris S220 to obtain DNA fragments averaging 4 kb in length. One-twentieth of the total chromatin served as input. The remaining material was used in the IP, which was performed using the HA-tag (C29F4) rabbit mAb conjugated to magnetic beads (Cell Signaling Technologies) at 4° C. overnight to pull down the HA-tagged dSpCas9 and interacting DNA. Beads were washed 7 times with wash buffer (50 mM Hepes [pH 7.6], 500 mM LiCl, 1 mM EDTA, 1% Igepal and 0.7% Na-Deoxycholate). Immunocomplexes were eluted from the beads with elution buffer (50 mM Tris-HCl, 10 mM EDTA and 1% SDS) at 65° C. for 15 minutes. Crosslinks in the IP and input were reversed overnight at 65° C. and treated with RNase A and proteinase K to remove RNAs and proteins. DNA was extracted with phenol-chloroform and precipitated with ethanol. The barcoded region associated with each repeat length was amplified from the isolated DNA fragments using primers containing adapters facilitating deep-sequencing.

For the RNA IP experiments, 48 hours after transfection, cells were harvested in 1 mL of lysis buffer (100 mM Kcl, 5 mM MgCl2, 10 mM HEPES [pH 7.0], 0.5% Igepal, 1 mM DTT, 100 U/mL SUPERase In RNase inhibitor (Thermo Fisher), 2 mM vanadyl ribonucleoside complexes solution, 25 uL/mL protease inhibitor cocktail). One-twentieth of the resulting lysate was used as input and the remaining lysate was incubated with HA-tag (C29F4) rabbit mAb conjugated to magnetic beads (Cell Signaling Technologies) at 4° C. overnight to pull down the HA-tagged dSpCas9 and interacting RNA. Beads were washed with the lysis buffer four times at 4° C. and immunocomplexes were eluted off with 0.1% SDS and proteins removed using proteinase K at 50° C. for 30 minutes. RNA was isolated using the Direct-zol RNA miniprep kit (Zymo Research) and contaminating DNA was eliminated using TURBO DNase (Thermo Fisher). cDNA was generated using Superscript IV Reverse Transcriptase (Thermo Fisher) and subsequently barcoded regions were amplified using flanking primers carrying sequences suitable for deep sequencing.

### FISH and Immunofluorescence (IF) Analyses

To detect nuclear RNA foci, cells or muscle fibers were fixed with 4% PFA for 10 minutes at room temperature followed by ice cold RNAse free 70% ethanol for 30 mins. Fixed samples were washed with a 25% formamide wash buffer at 30° C. for 30 mins and then hybridized with a CalFluor 610 conjugated (CAG)₁₀ oligonucleotide (Biosearch Technologies) in a 25% formamide hybridization buffer overnight at 30° C. Finally samples were washed two times at 30° C. with wash buffer for 30 minutes to an hour, incubated with DAPI (1 mg/mL) and mounted in Vectashield. Further IF analysis was performed on HeLa cells and DM1 myoblasts to detect the presence of the dCas9-HA protein. After excess oligonucleotide was washed off, the cells were blocked in 3% normal goat serum in 1% Triton X-100-PBS for 30 mins at room temperature, incubated with anti-HA antibody (1:500, #3724, Cell Signaling Technologies) overnight at 4° C., washed with 1×PBS, incubated with Alexa Fluor 488 conjugated anti-rabbit secondary antibody (1:500, Life Technologies) for 2 hours, washed and incubated with DAPI for 5 mins and mounted in Vectashield. Slides were imaged using the Zeiss LSM 880 Confocal Laser Scanning Microscope. IF analysis to detect dCas9 in mouse muscle fibers from the EDL of mice injected with AAV6-dSaCas9-(CAG) 6 was conducted as described above, except samples were fixed with 100% isopropanol at −20° C. for 10 mins.

Clcn1 was detected in mouse muscle by performing IF on frozen muscle sections of the TA. Frozen muscle was sectioned into 10 uM slices, fixed with 100% acetone at −20° C. for 20 minutes, washed with 0.3% Triton X-100-PBS and incubated with rabbit anti Clcn1 (1:100, #CLC11-S, Alpha Diagnostic International) overnight at 4° C. Samples were incubated with goat anti-rabbit Alexa Fluor 568 (1:500, Thermo Fisher) for 2 hours at RT and then treated with DAPI and mounted in Vectashield.

### Western Analyses for RAN Peptides

HEK293Tcells were transfected with one of the following plasmids that express tagged RAN translated products: pcDNA-6XStop-(CTG)₁₅₀-3X(FLAG-HA-cMyc-His), pcDNA-6XStop-(CCTG)₁₃₇-3X(FLAG-HA-cMyc-His) or pcDNA-6XStop-(G₄C₂)₁₂₀-3X(FLAG-HA-cMyc-His). These cells were co-transfected with the pXdCas9 plasmid expressing the dCas9 protein, and U6 expression vectors expressing the control gRNA or gRNA's targeting either strand of the CTG, CCTG and G₄C₂ repeats ((CAG)₆ or (CUG)₆ gRNAs, (CAGG)₅, (AGGC)₅, (GCAG)₅, or (CCUG)₅ gRNAs and (C₄G₂)₃ or (G₄C₂)₃ gRNAs, respectively).

72 hours after transfection, cells in each well of a 12-well tissue culture plate were gently rinsed 1× with PBS and lysed in 200 ul of RIPA Buffer (50 mM Tris-Cl pH 7.4, 150 mM NaCl, 0.1% Na-Deoxycholate, 1% NP-40, 0.5% SDS) with protease inhibitors for 30 minutes on ice. Genomic DNA was sheared by 8-10 passages through a 21-gauge needle. The resulting lysate was centrifuged at 18,000×g for 15 min and the supernatant was collected. The protein concentration of the lysate was determined using Pierce™ BCA Protein Assay. Equal amounts of protein were loaded and separated on a 4-12% NuPage Bis-Tris gel (Novex) and transferred to a nitrocellulose membrane (Amersham). The membrane was blocked in 5% milk in PBS-Tween20 (0.05%) for 1 hour and probed with anti-FLAG (1:2000) or anti-HA (1:1000) antibody in 1% milk solution in PBS-Tween20 (0.05%) overnight at 4° C. After the membrane was incubated with anti-mouse and anti-rabbit HRP (1:10,000) for 2 hours at room temperature, the bands were detected using the SuperSignal™ West Femto Maximum Sensitivity Substrate as per manufacturers protocol (Zu et al., 2011).

### Recombinant AAV Production

Viral production was achieved through transfection of HEK293T cells cultured in 150 mm plates with the pAAV6 serotype packaging plasmid (Rutledge et al., 1998), pXX6 helper plasmid that contains the adenovirus E4, VA and E2a helper regions (Xiao et al., 1998) and AAV2-ITR containing plasmid expressing dSaCas9 and the control or (CAG)₆ gRNA (generated from the SaCas9 plasmid pX601-AAV-CMV::NLS-SaCas9-NLS-3xHA-bGHpA; U6::BsaI-sgRNA, Addgene #61591). Transfections were carried out using the TransIT-LT1 transfection reagent and recommended protocols (MirusBio). Cells were harvested between 48 h and 72 h post-transfection, recombinant AAV2/6 virus was purified by iodixanol step gradients followed by vector concentration and buffer exchange with lactated Ringer's in an Apollo 150 kDa concentrator (Orbital Biosciences) (Zolotukhin et al., 2002). Virus titers were determined using the Quant-iT Picogreen dsDNA assay kit (Life Technologies) (Piedra et al., 2015) and found to be ˜10¹¹ vg/mL.

### Muscle Fiber Isolation

Single muscle fibers were isolated from 3-4 week old HSA^{LR} mice as described previously (Pasut et al., 2013). Briefly, the EDL was dissected and digested with a 0.2% Collagenase Type I in DMEM solution in a 37° C. water bath for 1 hour without agitation. The digested muscle was then flushed with DMEM to separate out individual muscle fibers. Fibers were cultured in DMEM containing 20% FBS overnight before infection with AAV.

### Transduction of Myoblasts and Muscle Fibers

Virus carrying dSaCas9 and control or (CAG)₆ gRNA was used to infect human DM1 primary myoblast cell lines and HSA^{LR} mouse EDL muscle fibers. To determine whether blocking expression of the CTG repeats in the human myoblasts affected the presence of RNA foci and splicing of MBNL targets, cells were grown to 60% confluency on CC2 chamber slides and infected for 6 days (3 days in growth media plus 3 days in differentiation media) with viral titers of 10⁹. To analyze the effects on RNA foci in HSA^{LR} muscle fibers, 10-20 muscle fibers were cultured in wells of a 96 well plate and infected with a 10⁹ viral titer for 48 h.

### Quantitating Signal Intensity of Nuclear Foci

Python scripts were written to quantitate intensity of FISH signal from RNA foci within nuclei of muscle fibers (python functions are listed below, and also see FIG. 16). Regions of interest were defined across each tile-scanned z-stack image obtained by confocal imaging. To identify and segment nuclei, 16-bit intensity values were scaled to lie between 0 and 1 (skimage.img_as_float). Then, a gaussian filter of standard deviation 3 pixels was applied to the raw DAPI signal (skimage.filters.gaussian), and a threshold of mean+5*standard deviation of the gaussian filtered signal was used to generate a binary mask. Holes were filled (scipy.ndimage.fill_holes), and a binary opening operation was performed to remove salt noise (skimage.morphology.binary_opening with ball structured element of radius 2). Nuclei were segmented and labeled (skimage.measure.label), and objects with pixel volume <1000 or >20000 were removed. FISH signal was scaled to lie between 0 and 1 as above, and a grayscale opening operation was performed to measure background (skimage.morphology.opening with a ball structured element of radius 3). This background intensity multipled by 3 was subtracted from the FISH signal to yield background-subtracted FISH signal. The binary nucleus mask was applied to this signal, and total intensity was measured within each nucleus, and divided by the nuclear volume in pixels, to obtain the final FISH signal per unit volume for each nucleus. This procedure was applied to all regions of interest across all fibers.

### Analysis of RNAseq Data

100 ng of RNA was used to prepare RNA-Seq libraries using the KAPA Ribo-Erase Strand-Specific kit. Samples were pooled and sequenced on the NextSeq 500 Version 2, using a High-Output 2×75 kit. Reads were mapped to hg19 by Hisat2, and splicing events were quantitated by MISO. Ψ values from DM tibialis biopsies were fit to sigmoid curves using 4-parameter estimation, where Ψ=Ψₘᵢₙ+(Ψₘₐₓ−Ψₘᵢₙ)/(1+e^{-slope*([MBNL]inferred-EC50)}), using python/scipy packages. The [MBNL]_{inferred} value was taken from Wagner et al (Wagner et al., 2016). The “fit error” was evaluated by taking the sum of squared errors between observed Ψ and Ψ as predicted by the sigmoid curves. Events consistently regulated between non-DM1 and DM1 myoblasts were identified using a modified monotonicity test (Wang et al., 2015), ΔΨ>0.1, BF>5) where the 2 non-DM1 libraries were grouped together, and 4 DM1 libraries were grouped together. For FIG. 11B, events with <1.3 sigmoid fit error and >1 monotonicity Z-score were selected for display. For FIG. 11F, only events identified in FIG. 11B to lie in the upper right or lower left quadrants were further analyzed; in addition, events were required to exhibit <0.33 difference in Ψ between cells treated with AAV-dSaCas9-control gRNA and non-DM1 tibialis biopsies. Raw RNA-Seq reads for these libraries are publicly available (GEO accession number pending).

### Electromyography

To determine whether expression of dCas9-(CAG)₆ rescued myotonia in the HSA^{LR} mice, mice were injected with AAV6-dSaCas9 and control or (CAG)₆ gRNA at 10¹⁰ viral genomes per mouse via the temporal vein at P2. Myotonia was assessed by electromyography (EMG) at 5 weeks of age as described previously (Kanadia et al., 2003). EMG was performed under general anaesthesia (intraperitoneal ketamine, 100 mg/kg; xylazine, 10 mg/kg) using 30 gauge concentric needle electrodes with at least 15 needle insertions per muscle in the hindlimb muscles, gastrocnemius and TA. Myotonic discharges were denoted as a percentage of the total number of insertions. In FIG. 11A, each point represents one of the hindlimb muscles from a single animal, but in some animals, both muscles were tested. N=2 mice for control gRNA and N=4 mice for (CAG) 6 gRNA.

### Data and Software Availability

The accession number for the data reported herein is GEO: GSE103997, which is incorporated herein by reference.

1. A method of treating a disease characterized by expanded microsatellite repeats, the method comprising administering to a subject in need thereof an effective amount of a composition that impedes or inhibits transcription of expanded microsatellite repeats.

2. The method of claim 1, wherein said composition comprises an adeno-associated virus (AAV) vector comprising a nucleic acid encoding a deactivated Cas9 (dCas9), an AAV virion comprising a nucleic acid encoding a dCas9, an AAV viral particle comprising a nucleic acid encoding a dCas9, a small molecule therapeutic, or a combination thereof.

3. The method of claim 2, wherein said composition further comprises a guide RNA (gRNA).

4. The method of claim 3, wherein said guide RNA comprises (CAG)₆ gRNA (SEQ ID NO:1), (CTG)₆ (SEQ ID NO:2) gRNA, (CUG)₆ (SEQ ID NO:3) gRNA, (AGC)₆ (SEQ ID NO:4) gRNA, (GCA)₆ (SEQ ID NO:5) gRNA, (CAGG)₅ (SEQ ID NO:6) gRNA, (AGGC)₅ (SEQ ID NO:7) gRNA, (GGCA)₅ (SEQ ID NO:8) gRNA, (GCAG)₅ (SEQ ID NO:9) gRNA, C₄G₂ gRNA, G₄C₂ gRNA, (C₄G₂)₃ (SEQ ID NO:10) gRNA, (G₄C₂)₃ (SEQ ID NO:11) gRNA, CAG repeat-targeting gRNA, CTG repeat-targeting gRNA, CCTG repeat-targeting gRNA, CAGG repeat-targeting gRNA, G₄C₂ repeat-targeting gRNA, C₄G₂ repeat-targeting gRNA, or a combination thereof.

5. The method of claim 2, wherein said small molecule therapeutic comprises actinomycin D, echinomycin, mythramycin A, or a combination thereof.

6. The method of claim 1, wherein said disease comprises myotonic dystrophy type 1, myotonic dystrophy type 2, C9ALS/FTD, spinocerebellar ataxias, Fuch's endothelial corneal dystrophy, or a neurological disorder.
